(19) United States
(12) Patent Application Publication
(10) Pub. No.: US 2026/0174026 A1
(43) Pub. Date: Jun. 25, 2026

(54) LETTUCE VARIETIES 45-IC7021 RZ (RICHARDS RZ), 45-IC7028 RZ (POLICE RZ) and 45-IC7037 RZ (ROLLINGS RZ)

(71) Applicant: RIJK ZWAAN ZAADTEELT EN ZAADHANDEL B.V., De Lier (NL)

(72) Inventor: Maria Helena Roosenboom-Kooijmans, De Lier (NL)

(21) Appl. No.: 19/425,179

(22) Filed: Dec. 18, 2025

Related U.S. Application Data
(60) Provisional application No. 63/752,175, filed on Jan. 31, 2025.
(60) Provisional application No. 63/913,489, filed on Nov. 7, 2025.

(30) Foreign Application Priority Data
Dec. 19, 2024 (WO) PCT/EP2024/087719

(51) Int. Cl. A01H 6/14; A01H 5/12

(52) U.S. Cl. CPC A01H 6/1472; A01H 5/12

(57) ABSTRACT

The present invention relates to a Lactuca sativa seed designated lettuce variety 45-IC7021 RZ, lettuce variety 45-IC7028 RZ, and lettuce variety 45-IC7037 RZ. The present invention also relates to a Lactuca sativa plant produced by growing the lettuce variety 45-IC7021 RZ, lettuce variety 45-IC7028 RZ, or lettuce variety 45-IC7037 RZ seed. The invention further relates to methods for producing the lettuce variety, represented by lettuce variety 45-IC7021 RZ, lettuce variety 45-IC7028 RZ, or lettuce variety 45-IC7037 RZ.

### RELATED APPLICATIONS AND INCORPORATION BY REFERENCE

This application claims benefit of and priority to U.S. provisional patent application Ser. No. 63/752,175 filed Jan. 31, 2025; U.S. provisional patent application 63/913,489 filed Nov. 7, 2025; and international patent application Serial No. PCT/EP2024/087719 filed Dec. 19, 2024.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and can be employed in the practice of the invention. More specifically, all referenced documents are incorporated by reference to the same extent as if each individual document was specifically and individually indicated to be incorporated by reference.

### FIELD OF THE INVENTION

The present invention relates to a new lettuce (Lactuca sativa) varieties designated lettuce variety 45-IC7021 RZ, lettuce variety 45-IC7028 RZ, and lettuce variety 45-IC7037 RZ.

Lettuce variety 45-IC7021 RZ exhibits a combination of traits including resistance to downy mildew (Bremia lactucae) races Bl:29EU, Bl:30EU, Bl:32EU, Bl:33EU, and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as mature leaves with medium to dense incisions of margin, and an ease of harvest phenotype.

Lettuce variety 45-IC7028 RZ exhibits a combination of traits including resistance to downy mildew (Bremia lactucae) races Bl:29EU to Bl:37EU, Bl:40EU, Bl:41EU and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as reduced wound-induced surface discoloration, mature leaves with a dense incisions of margin, and an ease of harvest phenotype.

Lettuce variety 45-IC7037 RZ exhibits a combination of traits including [resistance to downy mildew (Bremia lactucae) races Bl:29EU to Bl:37EU, Bl:40EU, Bl:41EU and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as reduced wound-induced surface discoloration, and an ease of harvest phenotype.

### BACKGROUND OF THE INVENTION

All cultivated forms of lettuce belong to the highly polymorphic species, Lactuca sativa, which is grown for its edible head and leaves. As a crop, lettuces are grown commercially wherever environmental conditions permit the production of an economically viable yield.

Lactuca sativa is in the Cichoreae tribe of the Asteraceae (Compositae) family. Lettuce is related to chicory, sunflower, aster, scorzonera, dandelion, artichoke and chrysanthemum. Sativa is one of about 300 species in the genus Lactuca.

Lettuce cultivars are susceptible to a number of pests and diseases such as downy mildew (Bremia lactucae). Every year this disease leads to millions of dollars of lost lettuce crop throughout the world. Downy mildew (Bremia lactucae) is highly destructive on lettuce grown at relatively low temperature and high humidity. Downy mildew is caused by a fungus, Bremia lactucae, which can be one of the following strains: Bl:29EU, Bl:30EU, Bl:31EU, Bl:32EU, Bl:33EU, Bl:34EU, Bl:35EU, Bl:36EU, Bl:37EU, Bl:38EU, Bl:39EU, Bl:40EU (Plantum NL, IBEB press release, “New races of Bremia lactucae, Bl:29, Bl:30 and Bl:31 identified and nominated”, August 2013; Plantum NL, IBEB press release, “A new race of Bremia lactucae, Bl:32 identified and nominated in Europe”, May 2015; Plantum NL, IBEB-EU press release, “A new race of Bremia lactucae, Bl:33EU identified and denominated in Europe”, May 2017; Plantum NL, IBEB-EU press release, “Two new races of Bremia lactucae, Bl:34EU and Bl:35EU identified and nominated in Europe”, July 2018; Plantum NL, IBEB-EU press release, “A new race of Bremia lactucae, Bl:36EU identified and denominated in Europe”, July 2019; Plantum NL, IBEB EU press release, “A new race of Bremia lactucae, Bl:37EU identified and denominated in Europe”, June 2021; IBEB-EU press release, “Three new races of Bremia lactucae, Bl: 38EU, Bl: 39 EU and Bl: 40 EU, July 2023; Plantum NL Press Release IBEB-EU 2024, “A new race of Bremia lactucae BL:41EU, identified and denominated in Europe, July 2024”); and Bl:7US, Bl:8US, Bl:9US and Bl:10US (Schettini, T. M., Legg, E. J., Michelmore, R. W., 1991). Insensitivity to metalaxyl in California populations of Bremia lactucae and resistance of California lettuce cultivars to downy mildew. Phytopathology 81(1). p. 64-70; Michelmore R. & Ochoa. O. “Breeding Crisphead Lettuce, In: California Lettuce Research Board, Annual Report 2005-2006, 2006, Salinas, California, pp. 55-68; bremia.ucdavis.edu/data/Bl_9US.pdf; www.rijkzwaanusa.com/news/denomination-of-new-official-downy-mildew-race-in-lettuce-for-western-us, July 2025).

Downy mildew causes pale, angular, yellow areas bounded by veins on the upper leaf surfaces. Sporulation occurs on the opposite surface of the leaves. The lesions eventually turn brown, and they can enlarge and coalesce. These symptoms typically occur first on the lower leaves of the lettuce, but under ideal conditions can move into the upper leaves of the head. When the fungus progresses to this degree, the head cannot be harvested. Less severe damage requires the removal of more leaves than usual, especially when the lettuce reaches its final destination.

Of the various species of aphids that feed on lettuce, the currant-lettuce aphid (Nasonovia ribisnigri) is the most destructive species because it feeds both on the leaves of the lettuce as well as the heart of the lettuce, making it difficult to control with conventional insecticides. The lettuce aphid feeds by sucking sap from the lettuce leaves. Although direct damage to the lettuce can be limited, its infestation has serious consequences because the presence of aphids makes lettuce unacceptable to consumers.

Fusarium root rot (wilt) of lettuce, caused by the soil borne fungus Fusarium oxysporum f.sp. lactucae, is an important disease of lettuce in warmer growing areas. It is found in the lettuce winter production areas of Arizona and California, and causes significant damage in the early plantings. In recent years, Fusarium root rot has also spread to the summer production area of the Salinas Valley. The fungus can penetrate plants through natural apertures or via wounds on the roots. Transmission of Fusarium oxysporum f.sp. lactucae can occur through infected seeds, infected transplants, or spread by soil as spores can remain viable in soil for long periods of time. Affected plants exhibit leaf yellowing and wilting. Crown tissue and upper roots display signs of reddish brown necrosis, followed by decaying. Leaf veins can also show vascular necrosis. Infected plants are stunted and often die, resulting in significant crop losses, and unmarketable product. Fusarium root rot can be prevented by soil sterilization, however, this is an expensive process.

The production of packaged salad mixes, for example, involves harvesting and processing of lettuce, which induces a strong wound response on the cut surfaces. Such a wound-induced response can lead to a rapid deterioration of the processed product. This deterioration is manifested as a brown or pink discoloration at, or adjacent to the wound surface. The brown or pink color that develops over time on the cut leaf surface makes the product highly unattractive to potential customers.

The wound-induced surface discoloration can in part be countered by reducing oxygen levels in the packaging. However, using specialized packaging is costly and may result in anaerobic conditions. Anaerobic conditions may reduce shelf life and may promote the growth of micro-organisms that produce a bad smell, reducing the overall attractiveness and taste of the product. Therefore, lettuce plants that exhibit reduced wound-induced discoloration of cut leaf surfaces is desirable. Such lettuce plants stay visually more attractive post-processing, and the need for reduced oxygen levels in the packaging of the cut leaves of these lettuce plants is minimized.

Iceberg lettuce is part of Lactuca sativa L. (Capitata Group) and is also indicated as crisphead lettuce, although crisphead also involves the Batavia subtype. Iceberg lettuce became one of the most popular lettuce types and its leaves with a crunchy texture are used, for instance, in salads or sandwiches, or are cooked.

Harvesting an iceberg lettuce head is labor intensive and time consuming as it is mostly done manually. One of the challenges of harvesting the head is that an iceberg lettuce head is covered by many large outer leaves. When harvesting the head, said head is cut from the stem and the outer leaves are removed by using a knife. Ideally, the stem is cut between the lower part of the stem, where the outer leaves are attached, and the upper part of the stem, where the head leaves are attached, and which upper part of the stem is part of the head. In most cases the cutting of the stem takes place below the ideal cutting position, leaving part of the outer leaves attached to the head. These remaining outer leaves are removed by a second cut with the knife.

In order to improve the harvestability of iceberg lettuce a fast and easy harvesting is desired. One type of iceberg lettuce plant that is easier to harvest is already known. This type of plant has a longer stem segment below the head. It is described that this phenotype makes it easier for the person harvesting the head to cut the longer stem segment between soil and head. However, the ideal cutting position remains equally small, and in most cases part of the outer leaves of this plant are still present on the harvested head and still need to be removed by using a knife. Another disadvantage of this type of iceberg is that the longer stem segment leads to less support of the outer leaves to the lettuce head, the head can tilt due to wind or due to one-sided pressure of the harvesting knife.

Citation or identification of any document in this application is not an admission that such document is available as prior art to the present invention.

### SUMMARY OF THE INVENTION

The present invention relates to an iceberg lettuce plant exhibiting an ease of harvest phenotype. The invention further relates to a cell of said plant and to a seed wherein the plant grown from the seed is an iceberg lettuce plant exhibiting an ease of harvest phenotype. The invention further relates to propagation material capable of developing into and/or being derived from an iceberg lettuce plant exhibiting an ease of harvest phenotype. The invention also relates to a method for growing an iceberg plant and harvesting an iceberg lettuce plant. The invention relates to markers for the identification of an iceberg lettuce plant exhibiting an ease of harvest phenotype, and to the use of said markers. The invention further relates to a method for selecting and a method for producing an iceberg lettuce plant exhibiting an ease of harvest phenotype.

Given the need expressed by relevant stakeholders for a lettuce variety which shows a combination of traits including among other the aforementioned traits, and specifically an ease of harvest phenotype, the present invention addresses this need by providing three new types of lettuce (Lactuca sativa) variety, designated 45-IC7021 RZ, 45-IC7028 RZ, and 45-IC7037 RZ.

The present invention provides 3 new lettuce (Lactuca sativa) varieties designated 45-IC7021 RZ, 45-IC7028 RZ, and 45-IC7037 RZ. Lettuce variety 45-IC7021 RZ exhibits a combination of traits including resistance to downy mildew (Bremia lactucae) races Bl:29EU, Bl:30EU, Bl:32EU, Bl:33EU, and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as mature leaves with medium to dense incisions of margin, and an ease of harvest phenotype.

Lettuce variety 45-IC7028 RZ exhibits a combination of traits including resistance to downy mildew (Bremia lactucae) races Bl:29EU to Bl:37EU, Bl:40EU, Bl:41EU and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as reduced wound-induced surface discoloration, mature leaves with a dense incisions of margin, and an ease of harvest phenotype.

Lettuce variety 45-IC7037 RZ exhibits a combination of traits including resistance to downy mildew (Bremia lactucae) races Bl:29EU to Bl:37EU, Bl:40EU, Bl:41EU and Bl:7US to Bl:9US, resistance to currant-lettuce aphid (Nasonovia ribisnigri) biotype Nr:0, resistance to Fusarium oxysporum f.sp. lactucae race 1 (Fol:1) and resistance to race 1 (Fol:1) and race 4 (Fol:4), as well as reduced wound-induced surface discoloration, and an ease of harvest phenotype.

Representative seed comprising, or providing, and having the heritable genetic information for these aforementioned combinations of traits present in lettuce variety 45-IC7028 RZ, or lettuce variety 45-IC7037 RZ, have been deposited with the National Collections of Industrial, Marine and Food Bacteria (NCIMB) in Wellheads Place, Dyce, Aberdeen AB21 7 GB, UK and have been assigned NCIMB Accession No. 44407, and NCIMB Accession No. 44408, respectively. Representative seed comprising, or providing, and having the heritable genetic information for these aforementioned combinations of traits present in lettuce variety 45-IC7021 RZ have been deposited with the NCIMB under Accession No. 44645.

The present invention provides seed of a lettuce (Lactuca sativa) variety designated as 45-IC7021 RZ, 45-IC7028 RZ, or 45-IC7037 RZ. A sample of seed of said lettuce varieties, have been deposited with the National Collections of Industrial, Marine and Food Bacteria (NCIMB) in Wellheads Place, Dyce, Aberdeen AB21 7 GB, UK and have been assigned NCIMB Accession No. 44645, NCIMB Accession No. 44407, and NCIMB Accession No. 44408, respectively.

In one embodiment, the invention provides a lettuce plant grown from the seed of lettuce (Lactuca sativa) variety 45-IC7021 RZ, lettuce variety 45-IC7028 RZ, or lettuce variety 45-IC7037 RZ.

In another embodiment, the invention provides a lettuce plant designated as lettuce variety 45-IC7021 RZ, lettuce variety 45-IC7028 RZ, or lettuce variety 45-IC7037 RZ, which is a plant grown from seed having been deposited under NCIMB Accession No. 44645, 44407, and 44408, respectively, or representative seed of which having been deposited under NCIMB Accession No. 44645, 44407, and 44408, respectively.

In one embodiment, the invention provides for a lettuce plant which comprises genetic information for exhibiting the aforementioned combination of traits or resistances, or for exhibiting all of the physiological and morphological characteristics of a plant of the invention (e.g., a plant having all of the physiological and morphological characteristics of a plant of the invention can be a plant grown from deposited seed), wherein the genetic information is as contained in a plant, a representative sample of seed of said variety having been deposited under NCIMB Accession No. 44645, 44407, and 44408, respectively.

The invention provides a lettuce plant of lettuce variety 45-IC7021 RZ exhibits a combination of traits including resistance to downy mildew (Bremia lactucae) races Bl:29EU, Bl:30EU, Bl:32EU, Bl:33EU, and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as mature leaves with medium to dense incisions of margin, and an ease of harvest phenotype, representative seed of which having been deposited under NCIMB Accession No. 44645, or representative seed of which can comprise genetic information for expression by the plant of the combination of traits or resistances having been deposited under NCIMB Accession No. 44645.

The invention provides a lettuce plant of lettuce variety 45-IC7028 RZ exhibiting a combination of traits including resistance to downy mildew (Bremia lactucae) races Bl:29EU to Bl:37EU, Bl:40EU, Bl:41EU and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as reduced wound-induced surface discoloration, mature leaves with a dense incisions of margin, and an ease of harvest phenotype, representative seed of which having been deposited under NCIMB Accession No. 44407, or representative seed of which can comprise genetic information for expression by the plant of the combination of traits or resistances having been deposited under NCIMB Accession No. 44407.

The invention provides a lettuce plant of lettuce variety 45-IC7037 RZ exhibiting a combination of traits including resistance to downy mildew (Bremia lactucae) races Bl:29EU to Bl:37EU, Bl:40EU, Bl:41EU and Bl:7US to Bl:9US, resistance to currant-lettuce aphid (Nasonovia ribisnigri) biotype Nr:0, resistance to Fusarium oxysporum f.sp. lactucae race 1 (Fol:1) and to race 4 (Fol:4), as well as reduced wound-induced surface discoloration, and an ease of harvest phenotype, representative seed of which having been deposited under NCIMB Accession No. 44408, or representative seed of which can comprise genetic information for expression by the plant of the combination of traits or resistances having been deposited under NCIMB Accession No. 44408.

In one embodiment, the invention provides for a lettuce plant exhibiting the aforementioned combination of traits or resistances, or for a lettuce plant exhibiting all the physiological and morphological characteristics of a plant of the invention, and having the genetic information for so exhibiting the combination of traits, or all the physiological and morphological characteristics of a plant of the invention (e.g., a plant having all of the physiological and morphological characteristics of a plant of the invention can be a plant grown from deposited seed), wherein the genetic information is as contained in a plant, a sample of seed of said variety having been deposited under NCIMB Accession No. 44645, 44407, and 44408, respectively.

In an embodiment of the present invention, there also is provided a lettuce plant or parts of a lettuce plant of the invention, which can include parts of a lettuce plant exhibiting the aforementioned combination of traits or resistances and can comprise genetic information for the plant and/or plant parts to exhibit the aforementioned combination of traits or resistances, or for exhibiting all the physiological and morphological characteristics of a plant of the invention (e.g., a plant exhibiting all of the physiological and morphological characteristics of a plant of the invention can be a plant grown from deposited seed), or parts of a lettuce plant having any or all of the mentioned resistance(s) and/or combination of traits including one or more or all morphological and physiological characteristics tabulated herein, including parts of lettuce variety 45-IC7021 RZ, lettuce variety 45-IC7028 RZ, or lettuce variety 45-IC7037 RZ, wherein the plant parts are involved in sexual reproduction, which include, without limitation, microspores, pollen, ovaries, ovules, embryo sacs or egg cells and/or wherein the plant parts are suitable for vegetative reproduction, which include, without limitation, cuttings, roots, stems, cells or protoplasts and/or wherein the plant parts are tissue culture of regenerable cells in which the cells or protoplasts of the tissue culture are derived from a tissue such as, for example and without limitation, leaves, pollen, embryos, cotyledon, hypocotyls, meristematic cells, roots, root tips, anthers, flowers, seeds or stems.

The plants of the invention from which such parts can come from include those wherein a sample of seed of which having been deposited under NCIMB Accession Nos. 44645, 44407, and 44408 or lettuce variety or cultivar designated 45-IC7021 RZ, lettuce variety 45-IC7028 RZ, or lettuce variety 45-IC7037 RZ, as well as seed from such a plant, plant parts of such a plant (such as those mentioned herein) and plants from such seed and/or progeny of such a plant, advantageously progeny exhibiting such combination of such traits, each of which, is within the scope of the invention; and such combination of traits.

In a further embodiment there is a plant regenerated from the above-described plant parts or regenerated from the above-described tissue culture. Advantageously such a plant has morphological and/or physiological characteristics of lettuce variety 45-IC7021 RZ, lettuce variety 45-IC7028 RZ, or lettuce variety 45-IC7037 RZ and/or of a plant grown from seed, a representative sample of seed of which having been deposited under NCIMB Accession No. 44645, 44407, or 44408—including without limitation such plants having all of the physiological and morphological characteristics of lettuce variety 45-IC7021 RZ, lettuce variety 45-IC7028 RZ, or lettuce variety 45-IC7037 RZ and/or of a plant grown from seed, a representative sample of seed of which having been deposited under NCIMB Accession No. 44645, 44407, or 44408, respectively.

Accordingly, in still a further embodiment, there is provided a lettuce plant having all of the physiological and morphological characteristics of lettuce variety 45-IC7021 RZ, lettuce variety 45-IC7028 RZ, or lettuce variety 45-IC7037 RZ, a representative sample of seed of which having been deposited under NCIMB Accession No. 44645, 44407, or 44408, respectively. Such a plant may be grown from the seeds, regenerated from the above-described plant parts, or regenerated from the above-described tissue culture. A lettuce plant having any of the aforementioned resistance(s), and one or more morphological or physiological characteristics recited or tabulated herein, and a lettuce plant advantageously having all of the aforementioned resistances and the characteristics recited and tabulated herein, are preferred. Parts of such plants—such as those plant parts above-mentioned—are encompassed by the invention.

In a further aspect, the invention provides a method of vegetatively propagating a plant of lettuce variety 45-IC7021 RZ, lettuce variety 45-IC7028 RZ, or lettuce variety 45-IC7037 RZ can comprise (a) collecting tissue capable of being propagated from a plant of lettuce 45-IC7021 RZ, lettuce variety 45-IC7028 RZ, or lettuce variety 45-IC7037 RZ, a representative sample of seed of said variety having been deposited under NCIMB Accession No. 44645, 44407, or 44408, respectively, and (b) cultivating the tissue to obtain proliferated shoots and rooting the proliferated shoots to obtain rooted plantlets. Optionally the invention can further comprise growing plants from the rooted plantlets. Plantlets and plants produced by these methods, are encompassed by the invention.

In one embodiment, there is provided a method for producing a progeny of lettuce variety 45-IC7021 RZ, lettuce variety 45-IC7028 RZ, or lettuce variety 45-IC7037 RZ which can comprise crossing a plant designated 45-IC7021 RZ, lettuce variety 45-IC7028 RZ, or lettuce variety 45-IC7037 RZ with itself or with another lettuce plant, harvesting the resultant seed, and growing said seed.

In a further embodiment, a progeny plant is provided which is produced by this method, wherein said progeny exhibits a combination of traits present in lettuce variety 45-IC7021 RZ, lettuce variety 45-IC7028 RZ, or lettuce variety 45-IC7037 RZ.

In another embodiment, a progeny plant is provided which is produced by the above method, wherein said progeny exhibits all the morphological and physiological characteristics of the lettuce variety designated as lettuce variety 45-IC7021 RZ, lettuce variety 45-IC7028 RZ, or lettuce variety 45-IC7037 RZ, a representative sample of seed of said variety having been deposited under NCIMB Accession No. 44645, 44407, or 44408, respectively.

In a further embodiment there is provided a progeny plant produced by sexual or vegetative reproduction, grown from seeds, regenerated from the above-described plant parts, or regenerated from the above-described tissue culture of the lettuce cultivar or a progeny plant thereof, a representative sample of seed of which having been deposited under NCIMB Accession No. 44645, 44407, and 44408. The progeny can have any of the aforementioned resistance(s), and one or more morphological or physiological characteristics recited or tabulated herein, and a progeny plant advantageously having all of the aforementioned resistances and the characteristics recited and tabulated herein, are preferred.

Progeny of the lettuce variety 45-IC7021 RZ, lettuce variety 45-IC7028 RZ, or lettuce variety 45-IC7037 RZ can be modified in one or more other characteristics, in which the modification is a result of, for example and without limitation, mutagenesis or transformation with a transgene.

In still another embodiment, the present invention provides progeny of lettuce variety 45-IC7021 RZ, lettuce variety 45-IC7028 RZ, or lettuce variety 45-IC7037 RZ produced by sexual or vegetative reproduction, grown from seed, regenerated from the above-described plant parts, or regenerated from the above-described tissue culture of the lettuce cultivar or a progeny plant thereof.

The invention moreover relates to progeny of a plant, a cell, a tissue, or a seed of the invention, which progeny comprises QTLs on chromosome 2, 3 and 7 as defined herein. Such progeny can in itself be a plant, a cell, a tissue, or a seed. The progeny can in particular be progeny of a plant of the invention, representative seeds of which were deposited under NCIMB 44645, NCIMB 44407, and NCIMB 44408. As used herein, progeny comprises the first and all further descendants from a cross with a plant of the invention, wherein a cross comprises a cross with itself or a cross with another plant, and wherein a descendant that is determined to be progeny comprises the QTLs on chromosome 2, 3 and 7 as defined herein. Descendants can be obtained through selfing and/or further crossing of the deposit. Progeny also encompasses material that is obtained by vegetative propagation or another form of multiplication.

The invention further relates to a method for producing a seed of a lettuce variety 45-IC7021 RZ-, lettuce variety 45-IC7028 RZ-, or lettuce variety 45-IC7037 RZ-derived lettuce plant can comprise (a) crossing a plant of lettuce variety 45-IC7021 RZ, lettuce variety 45-IC7028 RZ, or lettuce variety 45-IC7037 RZ, a representative sample of seed of which having been deposited under NCIMB Accession No. 44645, 44407, and 44408, respectively, with a second lettuce plant, and (b) whereby seed of a lettuce variety 45-IC7021 RZ-, lettuce variety 45-IC7028 RZ-, or lettuce variety 45-IC7037 RZ-derived lettuce plant forms. This method can further comprise (c) crossing a plant grown from lettuce variety 45-IC7021 RZ-, lettuce variety 45-IC7028 RZ-, or lettuce variety 45-IC7037 RZ-derived lettuce seed with itself or with a second lettuce plant to yield additional lettuce variety 45-IC7021 RZ-, lettuce variety 45-IC7028 RZ-, or lettuce variety 45-IC7037 RZ-derived lettuce seed, (d) growing the additional lettuce variety 45-IC7021 RZ-, lettuce variety 45-IC7028 RZ-, or lettuce variety 45-IC7037 RZ-derived lettuce seed of step (c) to yield additional lettuce variety 45-IC7021 RZ-, lettuce variety 45-IC7028 RZ-, or lettuce variety 45-IC7037 RZ-derived lettuce plants, and (e) repeating the crossing and growing of steps (c) and (d) for an additional 3-10 generations to generate further lettuce variety 45-IC7021 RZ-, lettuce variety 45-IC7028 RZ-, or lettuce variety 45-IC7037 RZ-derived lettuce plants, and (f) whereby seed of a lettuce variety 45-IC7021 RZ-, lettuce variety 45-IC7028 RZ-, or lettuce variety 45-IC7037 RZ-derived lettuce plant forms. A seed produced by this method and a plant grown from said seed also form part of the invention.

The invention further relates to germplasm of a plant of the invention. The germplasm is constituted by all inherited characteristics of an organism and according to the invention encompasses at least the resistance trait of the invention. The germplasm can be used in a breeding program for the development of plants that exhibit the ease of harvest phenotype. The use of germplasm that comprises the QTLs on chromosome 2, 3 and 7 as defined herein in breeding is also part of the present invention.

The invention also relates to a method of introducing at least one new trait into a plant of lettuce variety 45-IC7021 RZ, lettuce variety 45-IC7028 RZ, or lettuce variety 45-IC7037 RZ can comprise: (a) crossing a plant of lettuce variety 45-IC7021 RZ, lettuce variety 45-IC7028 RZ, or lettuce variety 45-IC7037 RZ, a representative sample of seed of which having been deposited under NCIMB Accession No. 44645, 44407, and 44408, respectively, with a second lettuce plant that can comprise at least one new trait to produce progeny seed, (b) harvesting and planting the progeny seed to produce at least one progeny plant of a subsequent generation, wherein the progeny plant can comprise the at least one new trait, (c) crossing the progeny plant with a plant of lettuce variety 45-IC7021 RZ, lettuce variety 45-IC7028 RZ, or lettuce variety 45-IC7037 RZ to produce backcross progeny seed, (d) harvesting and planting the backcross progeny seed to produce a backcross progeny plant, and (e) repeating steps (c) and (d) for at least three additional generations to produce a lettuce plant of variety lettuce variety 45-IC7021 RZ, lettuce variety 45-IC7028 RZ, or lettuce variety 45-IC7037 RZ can comprise at least one new trait and all of the physiological and morphological characteristics of a plant of lettuce variety 45-IC7021 RZ, lettuce variety 45-IC7028 RZ, or lettuce variety 45-IC7037 RZ, when grown in the same environmental conditions. A lettuce plant produced by this method also forms part of the invention.

Mutation breeding is another method of introducing new traits into lettuce varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines.

Mutations can be introduced randomly by means of one or more chemical compounds, such as ethyl methane sulphonate (EMS), nitrosomethylurea, hydroxylamine, proflavine, N-methyl-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast neutron exposure, X-rays, gamma irradiation, and/or by insertion of genetic elements, such as transposons, T-DNA, retroviral elements.

Mutations can also be introduced by more specific, targeted introduction of at least one modification by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

In addition to these random mutagenesis approaches, mutations may also be created by means of a targeted, site-directed mutagenesis approach. This involves the site-specific mutagenesis of pre-selected nucleotides or regions in a DNA sequence. Site-directed mutagenesis tools include, but are not limited to, zinc fingers (ZFNs) and Transcription Activator Like Effector Nucleases (TALENs)—which can both be designed to target specific nucleotide sequences by protein engineering—and CRISPR-Cas (Clustered Regularly Interspaced Short Palindromic Repeats). The latter system comprises a protein component and an RNA component, whereby the protein component remains unchanged and the RNA component is customized to recognize specific nucleotide sequences. The engineered protein-RNA complex binds to a target site that is located in the immediate vicinity of a suitable protospacer adjacent motif (PAM) site, where it induces a double strand break. The repair mechanism of the cell will then repair the double strand break, thereby making it again a target site for the CRISPR-Cas complex. In some cases the repair mechanism introduces a mistake while repairing the double strand break, thereby altering the site in a way that it is no longer recognized by the RNA component of the CRISPR-Cas complex. This typically introduces small insertions or deletions into the target sequence, which may lead to e.g. frame shifts in a protein coding sequence, and hence give rise to the functional inactivation of an encoded protein. The CRISPR-Cas technology also allows the introduction of larger insertions (with the help of recombination template sequences) or deletions in a genomic target site of choice.

In recent years, many different CRISPR enzymes have been developed into gene editing tools. These include, but are not limited to, Cas9, Cas12a (also known as Cpf1), Cms1, MAD7, C2c2, CasX, CasY, CasΦ, Casλ, CASMINI, SHARC, Cas-CLOVER, and OpenCRISPR-1. Each of these proteins has specific characteristics, and mutated versions have also been designed to further diversify the gene editing toolbox.

More advanced applications of CRISPR-Cas include, but are not limited to, base editing, prime editing, and PASTE. Base editing utilizes a Cas nickase linked to a deaminase enzyme, which is able to make specific point mutations (base changes) in a target DNA sequence to which it is guided by a customized guide RNA molecule (Komor et al., Nature 533:420-424, 2016). Prime Editing involves the rewriting of a specific target DNA sequence by a reverse transcriptase enzyme, which is linked to a Cas nickase that is guided to a specific target DNA sequence by a customized guide RNA molecule. A pegRNA template sequence is thereby used to alter the target DNA sequence according to the experimenter's design (Anzalone et al., Nature 576:149-157, 2019). PASTE (Programmable Addition via Site-specific Targeting Elements) utilizes a Cas nickase fused to a reverse transcriptase and a serine integrase to enable the integration of large DNA sequences at pre-selected target sites in an organism's DNA (Yarnall et al., Nature Biotech 41:500-512, 2022). See also U.S. Pat. Nos. 11,643,652; 11,447,770; 11,795,452 and 11,912,985 on Prime editing, and U.S. Pat. Nos. 11,572,556; 11,827,881; 11,834,658 and 11,952,571 on PASTE.

Once a desired trait is observed through random or site-directed mutagenesis the trait can then be incorporated into existing germplasm by traditional breeding techniques. Details of mutation breeding can be found in Principles of Cultivar Development by Fehr, Macmillan Publishing Company, 1993.

The invention further relates to a method of producing a plant of lettuce variety 45-IC7021 RZ, lettuce variety 45-IC7028 RZ, or lettuce variety 45-IC7037 RZ can comprise at least one new trait, the method can comprise introducing a mutation or transgene conferring the at least one new trait into a plant of lettuce variety 45-IC7021 RZ, lettuce variety 45-IC7028 RZ, or lettuce variety 45-IC7037 RZ, wherein a representative sample of seed of said variety has been deposited under NCIMB Accession No. 44645, 44407, and 44408, respectively. A lettuce plant produced by this method also forms part of the invention.

In still a further embodiment, the invention provides a method of producing a lettuce seed which can comprise crossing a male parent lettuce plant with a female parent lettuce plant and harvesting the resultant lettuce seed, in which the male parent lettuce plant or the female parent lettuce plant is a lettuce plant of the invention, e.g. a lettuce plant having all of the morphological or physiological characteristics tabulated herein, including a lettuce plant of lettuce variety 45-IC7021 RZ, lettuce variety 45-IC7028 RZ, or lettuce variety 45-IC7037 RZ, a representative sample of seed of which having been deposited under NCIMB Accession No. 44645, 44407, and 44408, respectively. The resultant lettuce seed produced by this method and the lettuce plant that is produced by growing said lettuce seed also forms part of the invention.

In still a further embodiment, the invention provides a method of producing a lettuce cultivar which exhibits all of the physiological and morphological characteristics of lettuce variety 45-IC7021 RZ, lettuce variety 45-IC7028 RZ, or lettuce variety 45-IC7037 RZ, a representative sample of seed of said variety having been deposited under NCIMB Accession No. 44645, 44407, and 44408, respectively.

The invention even further relates to a method of producing lettuce leaves as a food product which can comprise: (a) sowing a seed of lettuce variety 45-IC7021 RZ, lettuce variety 45-IC7028 RZ, or lettuce variety 45-IC7037 RZ, a representative sample of seed of which having been deposited under NCIMB Accession No. 44645, 44407, and 44408, respectively, (b) growing said seed into a harvestable lettuce plant and (c) harvesting lettuce leaves or heads from the plant. The invention further comprehends packaging and/or processing the lettuce plants, heads or leaves.

Also encompassed by the invention is a container that can comprise one or more lettuce plants of the invention for harvest of leaves.

Further encompassed by the invention is a method of determining the genotype of a plant of lettuce variety 45-IC7021 RZ, lettuce variety 45-IC7028 RZ, or lettuce variety 45-IC7037 RZ, a representative sample of seed of which has been deposited under NCIMB Accession No. 44645, 44407, and 44408, respectively, or a first generation progeny thereof, can comprise obtaining a sample of nucleic acids from said plant and comparing said nucleic acids to a sample of nucleic acids obtained from a reference plant, and detecting a plurality of polymorphisms between the two nucleic acid samples, wherein the plurality of polymorphisms are indicative of lettuce variety 45-IC7021 RZ, lettuce variety 45-IC7028 RZ, or lettuce variety 45-IC7037 RZ and/or give rise to the expression of any one or more, or all, of the physiological and morphological characteristics of lettuce variety 45-IC7021 RZ, lettuce variety 45-IC7028 RZ, or lettuce variety 45-IC7037 RZ of the invention.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)), or the EPO (Article 83 of the EPC), such that Applicants reserve the right and hereby disclose a disclaimer of any previously described product, process of making the product, or method of using the product.

It is noted that in this disclosure and particularly in the claims, terms such as “comprises”, “comprised”, and “comprising” and the like (e.g., “includes”, “included”, “including”, “contains”, “contained”, “containing”, “has”, “had”, “having”, etc.) can have the meaning ascribed to them in U.S. Patent law, i.e., they are open ended terms. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits. Similarly, the terms “consists essentially of” and “consisting essentially of” have the meaning ascribed to them in U.S. Patent law, e.g., they allow for elements not explicitly recited, but exclude elements that are found in the prior art or that affect a basic or novel characteristic of the invention. See also MPEP § 2111.03. In addition, the term “about” is used to indicate that a value includes the standard deviation of error for the device or method being employed to determine the value.

These and other embodiments are disclosed or are obvious from and encompassed by the following Detailed Description.

### DEPOSITS

The Deposits for lettuce variety 45-IC7028 RZ, and lettuce variety 45-IC7037 RZ with NCIMB, Ltd, Wellheads Place, Dyce, Aberdeen AB21 7 GB, UK, on Jul. 29, 2024, under NCIMB Accession numbers 44407, and 44408, respectively were made and accepted pursuant to the terms of the Budapest Treaty, and the Deposit for lettuce variety 45-IC7021 with NCIMB, Ltd, Wellheads Place, Dyce, Aberdeen AB21 7 GB, UK, on Aug. 14, 2025, under NCIMB Accession number 44645 was made and accepted pursuant to the terms of the Budapest Treaty. Upon issuance of a patent, all restrictions upon the deposits will be removed, and the deposits are intended to meet the requirements of 37 CFR §§ 1.801-1.809. The deposits will be irrevocably and without restriction or condition released to the public upon the issuance of a patent and for the enforceable life of the patent. The deposits will be maintained in the depository for a period of 30 years, or 5 years after the last request, or for the effective life of the patent, whichever is longer, and will be replaced if necessary, including if the deposits ever become unviable, during that period.

### BRIEF DESCRIPTION OF THE DRAWINGS

The following detailed description, given by way of example, but not intended to limit the invention solely to the specific embodiments described, can best be understood in conjunction with the accompanying drawings.

FIG. 1 is an illustration of six different shapes of the fourth leaf from a 20-day old seedling grown under optimal conditions.

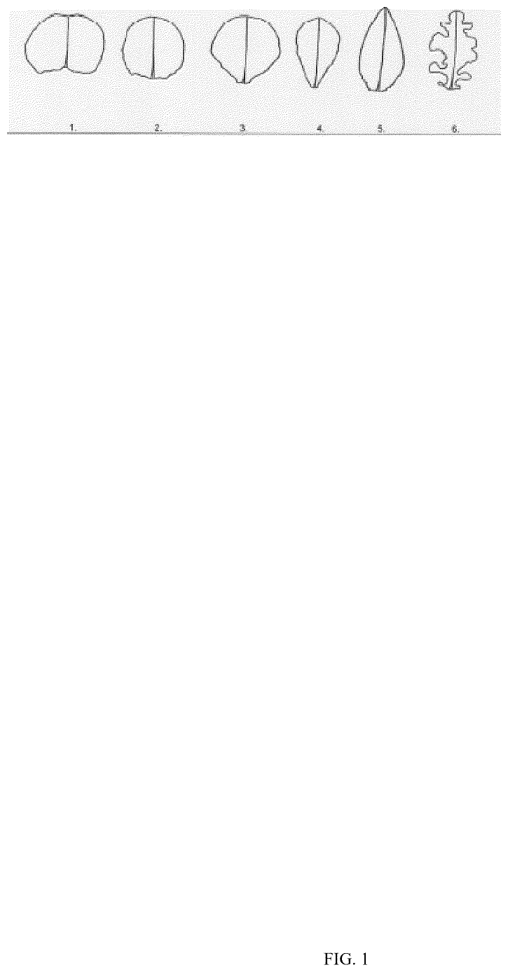

FIG. 2 is a plant and harvested heads of an iceberg plant of the invention.

FIG. 3 is a representation of the detachment of the outer leaf as described in Example 2.

### DETAILED DESCRIPTION OF THE INVENTION

The invention provides methods and compositions relating to plants, seeds and derivatives of a new lettuce variety herein referred to as lettuce variety 45-IC7021 RZ, lettuce variety 45-IC7028 RZ, or lettuce variety 45-IC7037 RZ. The lettuce variety 45-IC7021 RZ, lettuce variety 45-IC7028 RZ, and lettuce variety 45-IC7037 RZ are uniform and stable lines, distinct from other such lines.

In a preferred embodiment, the specific type of breeding method employed for developing a lettuce cultivar is pedigree selection, where both single plant selection and mass selection practices are employed. Pedigree selection, also known as the “Vilmorin system of selection,” is described in Fehr, W., Principles of Cultivar Development, Volume I, MacMillan Publishing Co., which is hereby incorporated by reference.

When pedigree selection is applied, in general selection is first practiced among F₂ plants. In the next season, the most desirable F₃ lines are first identified, and then desirable F₃ plants within each line are selected. The following season and in all subsequent generations of inbreeding, the most desirable families are identified first, then desirable lines within the selected families are chosen, and finally desirable plants within selected lines are harvested individually. A family refers to lines that were derived from plants selected from the same progeny from the preceding generation.

Using this pedigree method, two parents can be crossed using an emasculated female and a pollen donor (male) to produce F₁ offspring. Lettuce is an obligate self-pollination species, which means that pollen is shed before stigma emergence, assuring 100% self-fertilization. Therefore, in order to optimize crossing, a method of misting can be used to wash the pollen off prior to fertilization to assure crossing or hybridization.

Parental varieties are selected from commercial varieties that individually exhibit one or more desired phenotypes. Additionally, any breeding method involving selection of plants for the desired phenotype can be used in the method of the present invention.

The F₁ can be self-pollinated to produce a segregating F₂ generation. Individual plants can then be selected which represent the desired phenotype in each generation (F₃, F₄, F₅, etc.) until the traits are homozygous or fixed within a breeding population.

A detailed description of the development of lettuce variety 45-IC7021 RZ, lettuce variety 45-IC7028 RZ, and lettuce variety 45-IC7037 RZ is described in Table 1, Table 2, and Table 3, respectively. For lettuce variety 45-IC7021 RZ, the seedlot in year 6, seedlot 103299280, was deposited with the NCIMB as NCIMB Accession No. 44645. For lettuce variety 45-IC7028 RZ, the seedlot in year 5, seedlot S23R.3613, was deposited with the NCIMB as NCIMB Accession No. 44407. For lettuce variety 45-IC7037 RZ, the seedlot in year 5, seedlot S23R.3616, was deposited with the NCIMB as NCIMB Accession No. 44408.

TABLE 1
Breeding History 45-IC7021 RZ

| Year | Details |
|---|---|
| 2018 | F1 between internal RZ breeding line BL0250901 and internal RZ breeding line |
| | BL0256702 in glasshouse in Fijnaart, The Netherlands. No selection |
| 2019 | F1 plant grown for F2 seed production in glasshouse in Fijnaart, The |
| | Netherlands. No selection |
| 2019-2020 | F2 plant selected in open field, in Fijnaart, The Netherlands, followed by F3 |
| | seed production. Selection on various characteristics. |
| 2020-2021 | F3 plant selected in open field, in Fijnaart, The Netherlands, followed by F4 |
| | seed production. Selection on various characteristics. |
| 2021-2022 | F4 plant selected in open field, in Heijningen, The Netherlands, followed by F5 |
| | seed production. Selection on various characteristics. |
| 2022-2023 | F5 plant selected in open field, in Fijnaart, The Netherlands, followed by F6 |
| | seed production. Selection on various characteristics. |
| 2023 | F6 plant selected in plastic tunnel, in Daylesford, Australia, followed by F7 seed |
| | production. Selection on various characteristics. |
| 2024 | F7 line established uniform, multiplied in plastic tunnel, in Daylesford, |
| | Australia: seed lot: 103299280. Selection on uniformity and stability. |

TABLE 2
Breeding History 45-IC7028 RZ

| Year | Details |
|---|---|
| 2018 | F1 between internal RZ breeding line BL0250901 and internal RZ breeding line |
| | BL0256702 in glasshouse in Fijnaart, The Netherlands. No selection |
| 2019 | F1 plant grown for F2 seed production in glasshouse in Fijnaart, The |
| | Netherlands. No selection. |
| 2019-2020 | F2 plant selected in open field, in Fijnaart, The Netherlands, followed by F3 |
| | seed production. Selection on various characteristics. |
| 2020-2021 | F3 plant selected in open field, in Fijnaart, The Netherlands, followed by F4 |
| | seed production. Selection on various characteristics. |
| 2021-2022 | F4 plant selected in open field, in Heijningen, The Netherlands, followed by F5 |
| | seed production. Selection on various characteristics. |
| 2022-2023 | F5 plant selected in open field, in Fijnaart, The Netherlands, followed by F6 |
| | seed production. Selection on various characteristics. |
| 2023 | F6 plant selected in plastic tunnel, in Daylesford, Australia, followed by F7 seed |
| | production. Selection on various characteristics. |
| 2023-2024 | F7 plant selected in open field, in Fijnaart, The Netherlands, followed by F8 |
| | seed production. Selection on various characteristics. |
| 2024 | F8 line established uniform, multiplied in plastic tunnel, in Daylesford, |
| | Australia: seed lot: 23R.3613. Selection on uniformity and stability. |

TABLE 3
Breeding History 45-IC7037 RZ

| Year | Details |
|---|---|
| 2018 | F1 between internal RZ breeding line BL0250901 and internal RZ breeding line |
| | BL0256702 in glasshouse in Fijnaart, The Netherlands. No selection. |
| 2019 | F1 plant grown for F2 seed production in glasshouse in Fijnaart, The |
| | Netherlands. No selection. |
| 2019-2020 | F2 plant selected in open field, in Fijnaart, The Netherlands, followed by F3 |
| | seed production. Selection on various characteristics. |
| 2020-2021 | F3 plant selected in open field, in Fijnaart, The Netherlands, followed by F4 |
| | seed production. Selection on various characteristics. |
| 2021-2022 | F4 plant selected in open field, in Heijningen, The Netherlands, followed by F5 |
| | seed production. Selection on various characteristics. |
| 2022 | F5 plant selected in glasshouse, in Dinteloord, The Netherlands, followed by F6 |
| | seed production. Selection on various characteristics. |
| 2023 | F6 plant selected in glasshouse, in De Lier, The Netherlands, followed by F7 |
| | seed production. Selection on various characteristics. |
| 2024 | F7 plant selected in glasshouse, in De Lier, The Netherlands, followed by F8 |
| | seed production. Selection on various characteristics. |
| 2024 | F8 line established uniform, multiplied in glasshouse, in De Lier, The |
| | Netherlands: seed lot: 23R.3616. Selection on uniformity and stability. |

In one embodiment, a plant of the invention has all the physiological and morphological characteristics of lettuce variety 45-IC7021 RZ, lettuce variety 45-IC7028 RZ, or lettuce variety 45-IC7037 RZ. These characteristics of a lettuce plant of the invention, e.g. variety lettuce variety 45-IC7021 RZ, lettuce variety 45-IC7028 RZ, or lettuce variety 45-IC7037 RZ, are summarized in Tables 5, 6, and 7.

Next to the physiological and morphological characteristics mentioned in Tables 5, 6, and 7, a plant of the invention also exhibits an ease of harvest phenotype.

As used herein resistance against Bremia lactucae is defined as the ability of a plant to resist infection by each of the various strains Bl:29EU to Bl:40EU, and Bl:7US to Bl:10US of Bremia lactucae Regel. in all stages between the seedling stage and the harvestable plant stage. Bl:29EU to Bl:41EU means strains Bl:29EU, Bl:30EU, Bl:31EU, Bl:32EU, Bl:33EU, Bl:34EU, Bl:35EU, Bl:36EU, Bl:37EU, Bl:38EU, Bl:39EU, Bl:40EU, Bl:41EU (Van Ettekoven K, Van der Arend A J M, 1999. identification and denomination of ‘new’ races of Bremia lactucae. In: Lebeda A, Kristkova E (eds.) Eucarpia leafy vegetables '99. Palacky University, Olomouc, Czech Republic, 1999: 171-175; Van der Arend, A. J. M., Gautier, J., Guenard, M., Michel, H., Moreau, B., de Ruijter, J., Schut, J. W. and de Witte, I. (2003). Identification and denomination of ‘new’ races of Bremia lactucae in Europe by IBEB until 2002. In: Eucarpia leafy vegetables 2003. Proceedings of the Eucarpia Meeting on leafy vegetables genetics and breeding. Noordwijkerhout, The Netherlands. Eds. Van Hintum T., Lebeda A., Pink D., Schut J. pp 151-160; Van der Arend A J M, Gautier J, Grimault V, Kraan P, Van der Laan R, Mazet J, Michel H, Schut J W, Smilde D, De Witte I (2006) Identification and denomination of “new” races of Bremia lactucae in Europe by IBEB until 2006; incorporated herein by reference; Plantum NL, IBEB press release, “New race of Bremia lactucae Bl:27 identified and nominated”, May 2010; Plantum NL, IBEB press release, “New races of Bremia lactucae, Bl:29, Bl:30 and Bl:31 identified and nominated”, August 2013; Plantum NL, IBEB press release, “A new race of Bremia lactucae, Bl:32 identified and nominated in Europe”, May 2015; Plantum NL, IBEB-EU press release, “A new race of Bremia lactucae, Bl:33EU identified and denominated in Europe”, May 2017; Plantum NL, IBEB-EU press release, “Two new races of Bremia lactucae, Bl:34EU and Bl:35EU identified and nominated in Europe”, July 2018; Plantum NL, IBEB-EU press release, “A new race of Bremia lactucae, Bl:36EU identified and denominated in Europe”, July 2019; Plantum NL, IBEB EU press release, “A new race of Bremia lactucae, Bl:37EU identified and denominated in Europe, June 2021; IBEB-EU press release, “Three new races of Bremia lactucae, Bl: 38EU, Bl: 39EU and Bl: 40EU, July 2023; Plantum NL Press Release IBEB-EU 2024, “A new race of Bremia lactucae BL:41EU, identified and denominated in Europe, July 2024”). Bl:7US to Bl:10US means Bl:7US, Bl:8US, Bl:9US, Bl:10US (Schettini, T. M., Legg, E. J., Michelmore, R. W., 1991. Insensitivity to metalaxyl in California populations of Bremia lactucae and resistance of California lettuce cultivars to downy mildew, Phytopathology 81(1). p. 64-70; Michelmore R. & Ochoa. O. “Breeding Crisphead Lettuce.” In: California Lettuce Research Board, Annual Report 2005-2006, 2006, Salinas, California, pp. 55-68; bremia.ucdavis.edu/data/B1_9US.pdf, www.rijkzwaanusa.com/news/denomination-of-new-official-downy-mildew-race-in-lettuce-for-western-us, July 2025).

Resistance typically is tested by two interchangeable methods, described by Bonnier, F. J. M. et al. (Euphytica, 61(3):203-211, 1992; incorporated herein by reference). One method involves inoculating 7-day old seedlings, and observing sporulation 10 to 14 days later. The other method involves inoculating leaf discs with a diameter of 18 mm obtained from a non-senescent, fully grown true leaf and observing sporulation 10 days later.

As used herein, resistance against Nasonovia ribisnigri (Mosley), or currant-lettuce aphid, is defined as the plant characteristic which results in a non-feeding response of the aphid on the leaves of the plant in all stages between 5 true-leaf stage and harvestable plant stage (U.S. Pat. No. 5,977,443 to Jansen, J. P. A., “Aphid Resistance in Composites,” p. 12, 1999; incorporated herein by reference).

Resistance is tested by spreading at least ten aphids of biotype Nr:0 on a plant in a plant stage between 5 true leaves and harvestable stage, and observing the density of the aphid population on the plant as well as the growth reduction after 14 days in a greenhouse, with temperature settings of 23 degrees Celsius in daytime and 21 degrees Celsius at night. Day length is kept at 18 hours by assimilation lights.

As used herein, resistance against Fusarium oxysporum f.sp. lactucae, is defined as the ability of a plant to resist infection by race Fol:1. Resistance may be tested by two interchangeable methods. One method is described by Hubbard and Gerik (1993, Plant Disease 77(7): 750-754; incorporated herein by reference). The other test is described by Tsuchiya et al., (2004, J. Japan. Soc. Hort. Sci 73(2): 105-113; incorporated herein by reference).

As used herein, resistance against Fusarium oxysporum f.sp. lactucae race 4, is defined as the ability of a plant to resist infection by race Fol:4, also known as race 4. In 2015 a new variant of Fusarium in lettuce, Fusarium oxysporum f. sp. lactucae race 4, was identified and described by Gilardi et al. (2017). ISF has proposed a new differential set to their Disease Resistance Terminology Working Group (DRT WG) to differentiate this new race: worldseed.org/wp-content/uploads/2022/05/Heads-UP-2022_Differential_sets_Fol_race4.pdf After testing several isolates by all breeding companies active with lettuce, the isolate collected in the Netherlands and published by Gilardi et al., 2017 (a new race of Fusarium oxysporum f. sp. lactucae of lettuce. Plant Pathol. 66, 677-688. Doi: 10.1111/ppa.12616) was chosen as a reference isolate. The test protocol was used as described in worldseed.org/document/isf-final-report-fol-race-4-lettuce/.

As used herein, reduced wound-induced surface discoloration is defined as the ability of a lettuce plant, due to genetic information in its genome, to show a reduced or no discoloration of its wounded surface, as compared to a control lettuce plant of the same type and same color not showing the reduced wound-induced surface discoloration of its wounded surface. For comparison of this trait, the standard variety Crunchita can be used as a control plant for lettuce variety 45-IC7021 RZ, lettuce variety 45-IC7028 RZ, or lettuce variety 45-IC7037 RZ.

As used herein, the term “wound” is to mean the irreversible disturbance of the natural plant, tissue and/or cell structure by methods like cutting, punching, slicing, abrasion, squashing, breaking, peeling, crushing, pressing, slashing, grinding, fluid injection, osmotic shock, detaching, mowing, shredding, rubbing and tearing.

This trait can be tested by a leaf disc test in which a round leaf disc is punched from a fresh, fully-grown true leaf of a young plant, for example a plant at the 4-6 true leaf stage. The leaf discs of the plant to be tested and the control plant are incubated between two sheets of wetted filter paper for about 3 to about 7 days at about 5° C. to 7° C. (such as, but not limited to a 3-day incubation at about 5° C.), and the presence or absence of pink discoloration around each leaf disc at the wound surface is observed. A leaf disc taken from a lettuce plant showing a reduced wound-induced surface discoloration trait due to genetic information in its genome which is responsible for the trait, will not show a pink-colored discoloration (or only very faintly) at the edge of the wound surface, and is in any case less than the discoloration of a leaf disc taken from a control lettuce plant, for example a plant of the standard variety Crunchita, not showing the reduced wound-induced surface discoloration trait which will show pink-colored discoloration at the edge of the wound surface (for leaf disc test, see generally U.S. Pat. No. 8,809,631).

As used herein, the ease of harvest phenotype is a phenotype that entails that the leaves of the iceberg lettuce plant can just be bent until they detach as they break from the stem more easily than in a regular iceberg lettuce plant and said ‘ease of harvest’ trait is covered by international patent application Serial No. PCT/EP2024/087719 filed Dec. 19, 2024, incorporated herein by reference. The large outer leaves of an iceberg lettuce plant with an ease of harvest phenotype can be easily removed without using a knife, the leaves can just be bend until they detach as they break from the stem more easily than for a regular iceberg lettuce plant not having the ease of harvest phenotype. Thereby there is no need to use a knife for removing the leaves thus reducing the risk of damaging the head. The ease of harvest iceberg is also more suitable for mechanical harvesting of the iceberg lettuce head, because outer leaves of a regular iceberg plant usually remain on the head after being harvested mechanically and still have to be removed manually with a knife. In lettuce plants with the ease of harvest phenotype, the redundant outer leaves can easily be removed by peeling away the outer layers of the head.

The iceberg lettuce plant having an ease of harvest phenotype according to the invention has a height/width ratio of the head of 0.8 to 1.5, leaves with flabellate to semi-flabellate venation, and outer leaves that can be easily detached from the stem. The ease of harvest phenotype comprises the detachment of the 12^{th} leaf from the stem when peeled off.

As used herein iceberg is defined as a subtype of cultivated lettuce with closed head, which is round-shaped, and which head has a strong degree of overlapping of the upper part of the leaves. The leaf of the iceberg lettuce plant is thick and has no divisions or very few divisions as well as an undulation of margin which is absent to medium. The venation of the leaf is flabellate (“CPVO, Protocol for tests on distinctness, uniformity and stability, Lactuca sativa L.” Entry into force on 01/01/2024; cpvo.europa.eu/sites/default/files/documents/2024-04/lactuca-6.4_0.pdf).

The ease of harvest phenotype in an iceberg lettuce plant lies in that the leaves can just be bent until they detach as they break from the stem more easily than in a regular iceberg lettuce plant. This phenotype is caused by the presence of three QTLs, a QTL on chromosome 2, a QTL on chromosome 3 and a QTL on chromosome 7. As used herein, a QTL comprising a marker means that the marker is genetically linked to said QTL. These markers developed for the ‘ease of harvest’ phenotype can be found in the sequence tabulation provided herein, see Table 4.

As used herein, the height/width ratio of a lettuce head is measured when the non-bolting plant is at mature stage and when its head has a weight of at least 400 grams. The stem part within such a head should not exceed 8 cm. The width corresponds to the largest width of the head and the height corresponds to highest height of the head.

Measurements and observations on the plants as described herein are done on at least 20 plants for each genotype that grown under the same conditions. Preferably, the plant is grown outdoors under long-day conditions: sowing 75-85 days before the longest day in a mild mid-latitude climate with a warm summer (Koppen-classification: Csb or Cfb; McKnight & Hess, 2000. Physical Geography: A Landscape Appreciation. Upper Saddle River, NJ: Prentice Hall).

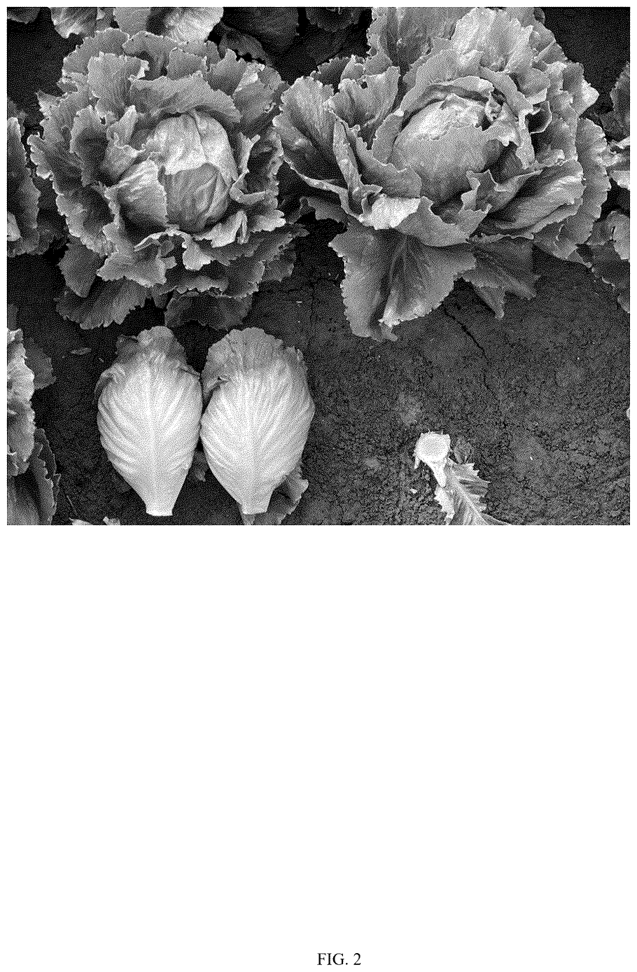

A head of the iceberg lettuce plant of the invention is shown in FIG. 2.

### Sequence Tabulation

The sequence information of the molecular markers linked to the three QTLs on chromosome 2, 3 and 7 is provided below in Table 4.

TABLE 4

| Marker | SEQ ID | | |
|---|---|---|---|
| name | NO | SNP | Sequence |
| LS03054 | SEQ ID | A | AACCATATTGTTCGGTCTTCATAACATTTTGGGGT |
| | NO: 1 | | TTTTGCGGCTTACTAAATTACTTGACGAAATTAGA |
| | | | GATGTAGTCATATTTTTTTTACATAGAGTTG |
| | SEQ ID | G | AACCATATTGTTCGGTCTTCATAACATTTTGGGGT |
| | NO: 2 | | TTTTGCGGCTTACTAGATTACTTGACGAAATTAGA |
| | | | GATGTAGTCATATTTTTTTTACATAGAGTTG |
| LS15311 | SEQ ID | C | CATGCCAAATGCTAAATGCCAAATAGCCAAAATAT |
| | NO: 3 | | GTTCAATCTATCATCCCTCTCCCTTTTATTATCTA |
| | | | TAGATTTTGCTCCAGATTTTCCTCTCCATCT |
| | SEQ ID | T | CATGCCAAATGCTAAATGCCAAATAGCCAAAATAT |
| | NO: 4 | | GTTCAATCTATCATCTCTCTCCCTTTTATTATCTA |
| | | | TAGATTTTGCTCCAGATTTTCCTCTCCATCT |
| LS15313 | SEQ ID | G | ACCATATGTCTAGCATACAAAATCGAAAATCATAA |
| | NO: 5 | | CTAACAACAAAGTTAGGATGTTACCTTTCAATTGT |
| | | | AGCTTGATCTTTAACCTTTGAGAGCTTTAGT |
| | SEQ ID | A | ACCATATGTCTAGCATACAAAATCGAAAATCATAA |
| | NO: 6 | | CTAACAACAAAGTTAAGATGTTACCTTTCAATTGT |
| | | | AGCTTGATCTTTAACCTTTGAGAGCTTTAGT |
| LS15315 | SEQ ID | A | AGATTTAGAATGAGAAACAGAATGGAGAATGAGA |
| | NO: 7 | | AGTATGAATTTTCATCAGTACTAGAATTCTGACC |
| | | | AAAATTTGGTTATATACATCCTCTTTCTTAAAA |
| | SEQ ID | G | AGATTTAGAATGAGAAACAGAATGGAGAATGAGA |
| | NO: 8 | | AGTATGAATTTTCATCGGTACTAGAATTCTGACC |
| | | | AAAATTTGGTTATATACATCCTCTTTCTTAAAA |
| LS15316 | SEQ ID | A | AAGGGATATAGAAGTGATTGGGGCTTCCAATGGAC |
| | NO: 9 | | ATTTTATGGAGTTTAAGGTCCTTGGACCACTCCCT |
| | | | TGGGAGTTTACGGCCGTAAACACATGAGTTT |
| | SEQ ID | C | AAGGGATATAGAAGTGATTGGGGCTTCCAATGGAC |
| | NO: 10 | | ATTTTATGGAGTTTACGGTCCTTGGACCACTCCCT |
| | | | TGGGAGTTTACGGCCGTAAACACATGAGTTT |
| LS15317 | SEQ ID | A | GGGGCACATATTGGATAATCTCGGAGTGATGGACT |
| | NO: 11 | | TGACTTTGTTACGCAACTCTCGTTTGAGTCCAACC |
| | | | ACTCTAGGGATGGGTCTTTTACTTGAAGAAT |
| | SEQ ID | G | GGGGCACATATTGGATAATCTCGGAGTGATGGAC |
| | NO: 12 | | TTGACTTTGTTACGCAGCTCTCGTTTGAGTCCAA |
| | | | CCACTCTAGGGATGGGTCTTTTACTTGAAGAAT |

| Marker | SEQ ID | | |
|---|---|---|---|
| name | NO | Indel | Sequence |
| LS14073 | SEQ ID | AACCACTACTGGAAA | GTTTTGGAGACAGCAACATCAT |
| | NO: 13 | ATTGGTAATAGAGTA | TAATACTAGCAGTAATAGTAAT |
| | | CACCCCCATCTAATAC | AATAACAACCACTACTGGAAA |
| | | GGTTGTAAAAAAACC | ATTGGTAATAGAGTACACCCC |
| | | CGTATTATAAAGGGA | CATCTAATACGGTTGTAAAAA |
| | | TAAATAAATATAATA | AACCCGTATTATAAAGGGATA |
| | | CACACAATTTTATGCA | AATAAATATAATACACACAAT |
| | | TGTAATCTAATACACC | TTTATGCATGTAATCTAATAC |
| | | CCTCTACTAATACGCC | ACCCCTCTACTAATACGCCCT |
| | | CTCTATGTACGTATTA | CTATGTACGTATTATATTCAC |
| | | TATTCACAATTTCAAC | AATTTCAACCGTACTCTATTA |
| | | CGTACTCTATTAACCT | ACCTTTTTCTAGTAGTGAACG |
| | | TTTTCTAGTAGTG | TTGGAGCACTATGTCTTCCTC |
| | | | CACCTCCTGTTTCATATAACA |
| | | | ACAACAACAA |
| | SEQ ID | — | GTTTTGGAGACAGCAACATCAT |
| | NO: 14 | | TAATACTAGCAGTAATAGTAAT |
| | | | AATAACAACGTTGGAGCACTAT |
| | | | GTCTTCCTCCACCTCCTGTTTC |
| | | | ATATAACAACAACAACAA |
| | SEQ ID | Sequence of | AACCACTACTGGAAAATTGGTA |
| | NO: 15 | insertion in | ATAGAGTACACCCCCATCTAAT |
| | | LS14073 | ACGGTTGTAAAAAAACCCGTAT |
| | | | TATAAAGGGATAAATAAATATA |
| | | | ATACACACAATTTTATGCATGT |
| | | | AATCTAATACACCCCTCTACTA |
| | | | ATACGCCCTCTATGTACGTATTA |
| | | | TATTCACAATTTCAACCGTACTC |
| | | | TATTAACCTTTTTCTAGTAGTG |

Thus, the invention relates to an iceberg lettuce plant exhibiting an ease of harvest phenotype, wherein said plant comprises a QTL on chromosome 3, wherein the QTL comprises marker LS15315 comprising a G at position 51 of SEQ ID NO: 8, or a G at position 51 of a sequence comprising at least 95% sequence identity to SEQ ID NO: 8, and marker LS15316 comprising a C at position 51 of SEQ ID NO: 10, or a C at position 51 of a sequence comprising at least 95% sequence identity to SEQ ID NO: 10, and marker LS15317 comprising a G at position 51 of SEQ ID NO: 12, or a G at position 51 of a sequence comprising at least 95% sequence identity to SEQ ID NO: 12; and wherein said lettuce plant further comprises a QTL on chromosome 2 and a QTL on chromosome 7, wherein the QTL on chromosome 2 comprises marker LS03054 comprising a G at position 51 of SEQ ID NO: 2, or a G at position 51 of a sequence comprising at least 95% sequence identity to SEQ ID NO: 2, and marker LS15311 comprising a T at position 51 of SEQ ID NO: 4, or a T at position 51 of a sequence comprising at least 95% sequence identity to SEQ ID NO: 4, and marker LS15313 comprising an A at position 51 of SEQ ID NO: 6, or an A at position 51 of a sequence comprising at least 95% sequence identity to SEQ ID NO: 6; and wherein the QTL on chromosome 7 comprises marker LS14073 comprising an insertion having SEQ ID NO: 15 at position 51 of SEQ ID NO:13, or an insertion having SEQ ID NO: 15 at position 51 of a sequence comprising at least 95% sequence identity to SEQ ID NO: 13.

In one embodiment the invention relates to an iceberg lettuce plant exhibiting an ease of harvest phenotype, wherein said plant comprises a QTL on chromosome 3, wherein the QTL comprises marker LS15315 comprising a G at position 51 of SEQ ID NO: 8, and marker LS15316 comprising a C at position 51 of SEQ ID NO: 10, and marker LS15317 comprising a G at position 51 of SEQ ID NO: 12; and wherein said lettuce plant further comprises a QTL on chromosome 2 and a QTL on chromosome 7, wherein the QTL on chromosome 2 comprises marker LS03054 comprising a G at position 51 of SEQ ID NO: 2, and marker LS15311 comprising a T at position 51 of SEQ ID NO: 4, and marker LS15313 comprising an A at position 51 of SEQ ID NO: 6; and wherein the QTL on chromosome 7 comprises marker LS14073 comprising an insertion having SEQ ID NO: 15 at position 51 of SEQ ID NO:13.

In one embodiment the plant of the invention comprises the QTLs on chromosome 2, 3 and 7 as defined herein homozygously.

The invention also relates to the use of the QTLs on chromosome 2, 3 and 7 as defined herein for producing an iceberg lettuce plant having an ease of harvest phenotype. The QTLs can be used for producing an iceberg lettuce plant by means of transgenesis or by introgression from a plant comprising the QTLs to a plant without the QTLs.

The invention further relates to an iceberg lettuce plant comprising the QTLs on chromosome 2, 3 and 7 as defined herein, wherein the QTLs are introgressed from a plant, from seed deposited with the NCIMB under deposit number NCIMB 44406, NCIMB 44407, or NCIMB 44408 or a progeny plant of seed of NCIMB accession NCIMB 44406, NCIMB 44407, or NCIMB 44408 that has retained said QTLs.

The characteristics of each of the deposits, lettuce variety 45-IC7021 RZ, lettuce variety 45-IC7028 RZ, and lettuce variety 45-IC7037 RZ are traits able to be introgressed into another plant (for details please refer to PCT/EP2024/087719 herein incorporated by reference).

Another aspect of the invention relates to a seed capable of growing into a plant of the invention wherein said plant comprises the QTLs on chromosome 2, 3 and 7 as defined herein. The invention also relates to use of said seed for the production of a plant of the invention, by growing said seed into a plant.

The invention also relates to a marker and/or the use thereof for the identification of the presence of the QTLs on chromosome 2, 3 and/or 7 as defined herein in an iceberg lettuce plant, wherein the marker is selected from any of the markers presented in the sequence tabulation.

The invention also relates to the use of markers as described herein for the identification of an iceberg lettuce plant comprising the ease of harvest phenotype.

This invention also relates to a method for selecting a plant of the invention, comprising identifying the presence of QTLs on chromosome 2, 3 and/or 7 as defined herein, and selecting a plant that comprises said QTL. Suitably, identifying the presence of any one of the QTLs on chromosome 2, 3 and 7 is done by using any one of the markers in the sequence tabulation as described herein. The identification or selection of a plant of the invention can also be done phenotypically as described herein.

The invention also relates to the use of markers as described herein for the identification of an iceberg lettuce plant comprising the ease of harvest phenotype. A marker can be defined as a reference sequence that comprises the modification(s) that can be detected using any suitable method known. The term “marker”, “molecular marker”, “genetic marker” or “DNA marker” refers to a feature of an organism's genome (e.g. a nucleotide or a polynucleotide sequence that is present in an organism's genome) that is associated with one or more loci of interest. A genetic marker is polymorphic in a population of interest. Genetic markers include, for example, single nucleotide polymorphisms (SNPs), indels (i.e. insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Genetic markers can, for example, be used to locate genetic loci containing alleles on a chromosome that contribute to variability of phenotypic traits. The term “marker” or “genetic marker” can also refer to a polynucleotide sequence complementary to a genomic sequence, such as a sequence of a nucleic acid used as a probe. The term “marker” then refers to a physical entity that can be used in molecular biological techniques for detecting the mutation.

In the context of the present invention, a marker listed in the sequence tabulation as described herein allows for the detection of the QTLs on chromosome 2, 3 and 7 as defined herein responsible for the ease of harvest phenotype, and the selection of iceberg lettuce plants that harbor in their genome the QTLs on chromosome 2, 3 and 7 as defined herein at any stage of their life cycle, even when the plants are only in the seedling stage. Marker-assisted breeding and selection greatly increases the speed with which a trait can be introduced into different genetic backgrounds, and with which it can be commercialized.

The markers listed in the sequence tabulation as described herein can also be used to develop other markers that are genetically linked to the QTLs on chromosome 2, 3 and 7 as defined herein.

Methods for detecting markers and specific alleles are abundantly known in the field. In general, these methods allow to distinguish between two different alleles of a marker, or the presence or absence of a marker on a specific chromosome. Detection of a polymorphism can be achieved by electrophoretic techniques, but the widespread availability of DNA sequencing often makes it easier to simply sequence amplified products directly. Once the polymorphic sequence difference is known, rapid assays for the detection of a polymorphism can be designed for progeny testing, generally involving some version of PCR amplification of specific alleles.

The invention further relates to a method for producing an iceberg lettuce plant comprising the ease of harvest phenotype, said method comprising:
a) crossing a plant of the invention with another plant to obtain a first generation population;
b) performing one or more rounds of selfing and/or crossing of the plant resulting from the cross to obtain a further generation population;
c) selecting from among the plants resulting from the further generation population of step b) a plant that comprises the QTLs on chromosome 2, 3 and 7 as defined herein, which plant comprises the ease of harvest phenotype.

In one embodiment, selecting a plant the invention is done by using one or more molecular markers genetically linked to the QTLs on chromosome 2, 3 and 7 as defined herein, preferably a marker in the sequence tabulation as described herein.

In another embodiment, a plant of the invention is phenotypically selected, in particular by determining the ease of harvest phenotype as defined herein.

In a specific embodiment the plant of the invention in step a) for producing an iceberg lettuce plant comprising the ease of harvest phenotype is a plant grown from seed deposited under NCIMB accession number NCIMB 44406, NCIMB 44407, or NCIMB 44408, or a progeny plant thereof that has retained the QTLs on chromosome 2, 3 and 7 as defined herein.

The invention further relates to a hybrid iceberg lettuce seed and to a method for the production of a hybrid iceberg lettuce seed comprising crossing a first parent plant with a second parent plant and harvesting the resultant hybrid seed, wherein the first parent plant and/or the second parent plant is a plant comprising the QTLs on chromosome 2, 3 and 7 as defined herein.

In a particular embodiment, the first and/or second parent plant is a plant grown from seed deposited under NCIMB accession number NCIMB 44406, NCIMB 44407, or NCIMB 44408, or a progeny plant thereof that has retained the QTLs on chromosome 2, 3 and 7 as defined herein.

In a particular embodiment the iceberg lettuce plant is grown from a seed deposited under NCIMB accession number NCIMB 44406, NCIMB 44407, or NCIMB 44408, or a progeny plant thereof that has retained the QTLs on chromosome 2, 3 and 7 as defined herein.

Embodiments of the inventions advantageously have one or more, and most advantageously all, of these characteristics.

In Table 5, the characteristics of lettuce variety 45-IC7021 RZ” are shown (Exhibit C—USDA Descriptors).

In Table 6 the characteristics of lettuce variety 45-IC7028 RZ are shown.

In Table 7, the characteristics of lettuce variety 45-IC7037 RZ are shown.

The information presented in the tables in Tables 5, 6 and 7 was determined in trial experiments in accordance with official Dutch plant variety registration authorities (Naktuinbouw).

The terminology and descriptors used by the Naktuinbouw, are in line with the descriptors of the “UPOV Guidelines for the Conduct of Tests for Distinctness, Uniformity, and Stability”, or the “Test Guidelines” for Lettuce. For the lettuce in these tables the version is TG/13/11 Rev. 3, adopted in Aug. 9 2024 was the standard. The terminology and descriptors used in these tables are in line with the official terminology as of the filing date, and are thus clear for a person skilled in the art.

Additionally, the definitions for resistance indicated in Tables 5-7 follow the guidelines presented in worldseed.org/document/definitions-of-the-terminology-plants-pests-v-o-seed-industry-2022/, which defines two levels of resistance. High resistance (HR): plant varieties that highly restrict the growth and/or development of the specified pest and/or the damage it causes under normal pest pressure when compared to susceptible varieties. These plant varieties may, however, exhibit some symptoms or damage under heavy pest pressure. Intermediate resistance (IR): plant varieties that restrict the growth and/or development of the specified pest and/or the damage it causes but may exhibit a greater range of symptoms or damage compared to high resistant varieties. Intermediate resistant plant varieties will still show less severe symptoms or damage than susceptible plant varieties when grown under similar environmental conditions and/or pest pressure.

As used herein, the terms “resistance” and “high resistance” and the adjectives “resistant” and “highly resistant” are used interchangeably. The term “intermediate resistance” and the adjective “intermediate resistant” is explicitly mentioned whenever applicable.

TABLE 5

| Variety | 45-IC7021 RZ |
|---|---|
| Type | Iceberg |
| Resistances HR | Bl: 29, 30, 32, 33EU/Bl: 7, 8, |
| | 10US/Fol: 1, Fol: 4/Nr: 0 |
| Reduced Wound-induced | Absent |
| Surface Discoloration | |
| Seed: Color | White |
| Leaf: Number of divisions | Absent or very few |
| Leaf: Anthocyanin coloration | Absent or very weak |
| Leaf: Color | Green |
| Leaf: Intensity of Green Color | Medium to Dark |
| Plant: Degree of overlapping | Medium to Strong |
| of upper part of leaves | |
| Plant: Head Diameter | Medium to large |
| Head: Size | Medium to large |
| Head: Shape in Llongitudinal Section | Broad elliptic |
| Head: Density | Dense to very Dense |
| Leaf: Glossiness of Upper Side | Very weak to Weak |
| Leaf: Density of Incisions of Margin | Medium to Dense |
| Time of Harvest Maturity | Medium to Late |
| Bolting | Very Late |
| Auxilliary Sprouting | Absent or Weak |
| Leaf: Attitude | Erect to Semi-Erect |
| Leaf: Shape | Obovate |
| Leaf: Shape of Apex | Rounded |
| Leaf: Longitudinal Section | Flat |
| Leaf: Thickness | Thick |
| Leaf: Blistering | Weak |
| Leaf: Size of Blisters | Small |
| Leaf: Undulation of Margin | Weak |
| Leaf: Type of Incisions of Margin | Crenate |
| Leaf: Depth of Incisions of Margin | Very shallow to Shallow |
| Leaf: Venation | Flabellate |
| Cotyledon to Fourth Leaf Stage: | Intermediate |
| Shape of Cotyledons | |
| Cotyledon to Fourth Leaf Stage: | Elongated |
| Shape of Fourth Leaf | |
| Plant Head Diameter | 18 cm |
| Spread of Frame Leaves | 35 cm |
| Butt Shape | Rounded |
| Butt Midrib | Moderately Raised |

TABLE 6

| Variety | 45-IC7028 RZ |
|---|---|
| Type | Iceberg |
| Resistances HR | Bl: 29-37, 40, 41EU/Bl: 7- |
| | 10US/Fol: 1, Fol: 4/Nr: 0 |
| Reduced Wound-induced | Present |
| Surface Discoloration | |
| Seed: Color | White |
| Leaf: Number of divisions | Absent or very few |
| Leaf: Anthocyanin coloration | Absent or very weak |
| Leaf: Color | Green |
| Leaf: Intensity of green color | Medium to Dark |
| Plant: Degree of overlapping of | Medium to strong |
| upper part of leaves | |
| Plant: Head diameter | Medium to Large |
| Head: Size | Medium |
| Head: Shape in longitudinal section | Broad Elliptic |
| Head: Density | Very Dense |
| Leaf: Glossiness of Upper Side | Weak to Medium |
| Leaf: Density of Incisions of Margin | Dense |
| Time of Harvest Maturity | Late |
| Bolting | Very Late |
| Auxilliary Sprouting | Absent or Weak |
| Leaf: Attitude | Semi-Erect |
| Leaf: Shape | Obovate |
| Leaf: Shape of Apex | Rounded |
| Leaf: Longitudinal section | Flat |
| Leaf: Thickness | Thick |
| Leaf: Blistering | Weak |
| Leaf: Size of blisters | Small |
| Leaf: Undulation of margin | Weak |
| Leaf: Type of Incisions of Margin | Irregularly Dentate |
| Leaf: Depth of Incisions of Margin | Very Shallow to Shallow |
| Leaf: Venation | Flabellate |
| Cotyledon to Fourth Leaf Stage: | Intermediate |
| shape of cotyledons | |
| Cotyledon to Fourth Leaf Stage: | Elongated |
| shape of fourth leaf | |
| Plant Head Diameter | 13 cm |
| Spread of Frame Leaves | 33 cm |
| Butt Shape | Rounded |
| Butt Midrib | Moderately Raised |

TABLE 7

| Variety | 45-IC7037 RZ |
|---|---|
| Type | Iceberg |
| Resistances HR | Bl: 29-37, 40, 41EU/Bl: 7- |
| | 10US/Fol: 1, Fol: 4/Nr: 0 |
| Reduced Wound-induced | Present |
| Surface Discoloration | |
| Seed: Color | White |
| Leaf: Number of divisions | Absent or very few |
| Leaf: Anthocyanin coloration | Absent or very weak |
| Leaf: Color | Green |
| Leaf: Intensity of Green Color | Medium to Dark |
| Plant: Degree of overlapping | Medium to Strong |
| of upper part of leaves | |
| Plant: Head Diameter | Medium to Large |
| Head: Size | Medium to Large |
| Head: Shape in longitudinal section | Broad Elliptic |
| Head: Density | Very Dense |
| Leaf: Glossiness of upper side | Weak |
| Leaf: Density of Incisions of Margin | Medium to Dense |
| Time of Harvest Maturity | Late |
| Bolting | Very Late |
| Auxilliary Sprouting | Medium to Strong |
| Leaf: Attitude | Semi-Erect |
| Leaf: Shape | Medium Oblate to Obovate |
| Leaf: Shape of Apex | Rounded |
| Leaf: Longitudinal section | Flat |
| Leaf: Thickness | Thick |
| Leaf: Blistering | Weak |
| Leaf: Size of blisters | Very Small to Small |
| Leaf: Undulation of margin | Weak |
| Leaf: Type of incisions of margin | Crenate |
| Leaf: Depth of incisions of margin | Very Shallow to Shallow |
| Leaf: Venation | Flabellate |
| Cotyledon to Fourth Leaf Stage: | Intermediate |
| shape of cotyledons | |
| Cotyledon to Fourth Leaf Stage: | Elongated |
| shape of fourth leaf | |
| Plant Head Diameter | 16 cm |
| Spread of Frame Leaves | 38 cm |
| Butt Shape | Rounded |
| Butt Midrib | Prominently Raised |

In one aspect the invention provides a new type of lettuce (Lactuca sativa) variety, designated lettuce variety 45-IC7021 RZ, lettuce variety 45-IC7028 RZ, or lettuce variety 45-IC7037 RZ.

Lettuce variety 45-IC7021 RZ exhibits a combination of traits including resistance to downy mildew (Bremia lactucae) races Bl:29EU, Bl:30EU, Bl:32EU, Bl:33EU, and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as mature leaves with medium to dense incisions of margin, and an ease of harvest phenotype.

Lettuce variety 45-IC7028 RZ exhibits a combination of traits including resistance to downy mildew (Bremia lactucae) races Bl:29EU to Bl:37EU, Bl:40EU, Bl:41EU and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as reduced wound-induced surface discoloration, mature leaves with a dense incisions of margin, and an ease of harvest phenotype.

Lettuce variety 45-IC7037 RZ exhibits a combination of traits including [resistance to downy mildew (Bremia lactucae) races Bl:29EU to Bl:37EU, Bl:40EU, Bl:41EU and Bl:7US to Bl:9US, resistance to currant-lettuce aphid (Nasonovia ribisnigri) biotype Nr:0, resistance to Fusarium oxysporum f.sp. lactucae race 1 (Fol:1) and race 4 (Fol:4), as well as reduced wound-induced surface discoloration, and an ease of harvest phenotype.

In an embodiment, the invention relates to lettuce plants that have all the physiological and morphological characteristics of the invention and have acquired said characteristics by introduction of the genetic material or information that is responsible for the characteristics from a suitable source, either by conventional breeding, or genetic modification, in particular by cisgenesis or transgenesis. Cisgenesis is genetic modification of plants with a natural gene, coding for an (agricultural) trait, from the crop plant itself or from a sexually compatible donor plant. Transgenesis is genetic modification of a plant with a gene from a non-crossable species or a synthetic gene.

Just as useful traits that can be introduced by backcrossing, useful traits can be introduced directly into the plant of the invention, being a plant of lettuce variety 45-IC7021 RZ, lettuce variety 45-IC7028 RZ, or lettuce variety 45-IC7037 RZ, by genetic transformation techniques; and, such plants of lettuce variety 45-IC7021 RZ, lettuce variety 45-IC7028 RZ, or lettuce variety 45-IC7037 RZ that have additional genetic information introduced into the genome or that express additional traits by having the DNA coding there for introduced into the genome via transformation techniques, are within the ambit of the invention, as well as uses of such plants, and the making of such plants.

Genetic transformation can therefore be used to insert a selected transgene into the plant of the invention, being a plant of lettuce variety 45-IC7021 RZ, lettuce variety 45-IC7028 RZ, or lettuce variety 45-IC7037 RZ or can, alternatively, be used for the preparation of transgenes which can be introduced by backcrossing. Methods for the transformation of plants, including lettuce, are well known to those of skill in the art.

Vectors used for the transformation of lettuce cells are not limited so long as the vector can express an inserted DNA in the cells. For example, vectors which can comprise promoters for constitutive gene expression in lettuce cells (e.g., cauliflower mosaic virus 35S promoter) and promoters inducible by exogenous stimuli can be used. Examples of suitable vectors include pBI binary vector. The “lettuce cell” into which the vector is to be introduced includes various forms of lettuce cells, such as cultured cell suspensions, protoplasts, leaf sections, and callus. A vector can be introduced into lettuce cells by known methods, such as the polyethylene glycol method, polycation method, electroporation, Agrobacterium-mediated transfer, particle bombardment and direct DNA uptake by protoplasts. To effect transformation by electroporation, one can employ either friable tissues, such as a suspension culture of cells or embryogenic callus or alternatively one can transform immature embryos or other organized tissue directly. In this technique, one would partially degrade the cell walls of the chosen cells by exposing them to pectin-degrading enzymes (pectolyases) or mechanically wound tissues in a controlled manner.

A particularly efficient method for delivering transforming DNA segments to plant cells is microprojectile bombardment. In this method, particles are coated with nucleic acids and delivered into cells by a propelling force. Exemplary particles include those which can be comprised of tungsten, platinum, and preferably, gold. For the bombardment, cells in suspension are concentrated on filters or solid culture medium. Alternatively, immature embryos or other target cells can be arranged on solid culture medium. The cells to be bombarded are positioned at an appropriate distance below the macroprojectile stopping plate. An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target lettuce cells. The screen disperses the particles so that they are not delivered to the recipient cells in large aggregates. It is believed that a screen intervening between the projectile apparatus and the cells to be bombarded reduces the size of projectiles aggregate and can contribute to a higher frequency of transformation by reducing the damage inflicted on the recipient cells by projectiles that are too large. Microprojectile bombardment techniques are widely applicable, and can be used to transform virtually any plant species, including a plant of lettuce variety 45-IC7021 RZ, lettuce variety 45-IC7028 RZ, or lettuce variety 45-IC7037 RZ.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Agrobacterium transformation vectors are capable of replication in E. coli as well as Agrobacterium, allowing for convenient manipulations. Moreover, advances in vectors for Agrobacterium-mediated gene transfer have improved the arrangement of genes and restriction sites in the vectors to facilitate the construction of vectors capable of expressing various polypeptide coding genes. The vectors have convenient multi-linker regions flanked by a promoter and a polyadenylation site for direct expression of inserted polypeptide coding genes. Additionally, Agrobacterium containing both armed and disarmed Ti genes can be used for transformation. In those plant strains where Agrobacterium-mediated transformation is efficient, it is the method of choice because of the facile and defined nature of the gene locus transfer. The use of Agrobacterium-mediated plant integrating vectors to introduce DNA into plant cells, including lettuce plant cells, is well known in the art (See, e.g., U.S. Pat. Nos. 7,250,560 and 5,563,055).

Transformation of plant protoplasts also can be achieved using methods based on calcium phosphate precipitation, polyethylene glycol treatment, electroporation, and combinations of these treatments.

A number of promoters have utility for plant gene expression for any gene of interest including but not limited to selectable markers, scoreable markers, genes for pest tolerance, disease resistance, nutritional enhancements and any other gene of agronomic interest. Examples of constitutive promoters useful for lettuce plant gene expression include, but are not limited to, the cauliflower mosaic virus (CaMV) P-35S promoter, a tandemly duplicated version of the CaMV 35S promoter, the enhanced 35S promoter (P-e35S), the nopaline synthase promoter, the octopine synthase promoter, the figwort mosaic virus (P-FMV) promoter (see U.S. Pat. No. 5,378,619), an enhanced version of the FMV promoter (P-eFMV) where the promoter sequence of P-FMV is duplicated in tandem, the cauliflower mosaic virus 19S promoter, a sugarcane bacilliform virus promoter, a commelina yellow mottle virus promoter, the promoter for the thylakoid membrane proteins (psaD, psaF, psaE, PC, FNR, atpC, atpD, cab, rbcS) (see U.S. Pat. No. 7,161,061), the CAB-1 promoter (see U.S. Pat. No. 7,663,027), the promoter from maize prolamin seed storage protein (see U.S. Pat. No. 7,119,255), and other plant DNA virus promoters known to express in plant cells. A variety of plant gene promoters that are regulated in response to environmental, hormonal, chemical, and/or developmental signals can be used for expression of an operably linked gene in plant cells, including promoters regulated by (1) heat, (2) light (e.g., pea rbcS-3A promoter, maize rbcS promoter, or chlorophyll a/b-binding protein promoter), (3) hormones, such as abscisic acid, (4) wounding (e.g., wunl, or (5) chemicals such as methyl jasmonate, salicylic acid, or Safener. It can also be advantageous to employ organ-specific promoters.

Exemplary nucleic acids which can be introduced to the lettuce variety of this invention include, for example, DNA sequences or genes from another species, or even genes or sequences which originate with or are present in lettuce species, but are incorporated into recipient cells by genetic engineering methods rather than classical reproduction or breeding techniques. However, the term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene can already be present in such a cell. The type of DNA included in the exogenous DNA can include DNA which is already present in the plant cell, DNA from another plant, DNA from a different organism, or a DNA generated externally, such as a DNA sequence containing an antisense message of a gene, or a DNA sequence encoding a synthetic or modified version of a gene.

Many hundreds if not thousands of different genes are known and could potentially be introduced into a plant of lettuce variety 45-IC7021 RZ, lettuce variety 45-IC7028 RZ, or lettuce variety 45-IC7037 RZ. Non-limiting examples of particular genes and corresponding phenotypes one can choose to introduce into a lettuce plant include one or more genes for insect tolerance, pest tolerance such as genes for fungal disease control, herbicide tolerance, and genes for quality improvements such as yield, nutritional enhancements, environmental or stress tolerances, or any desirable changes in plant physiology, growth, development, morphology or plant product(s).

Alternatively, the DNA coding sequences can affect these phenotypes by encoding a non-translatable RNA molecule that causes the targeted inhibition of expression of an endogenous gene, for example via antisense- or cosuppression-mediated mechanisms. The RNA could also be a catalytic RNA molecule (i.e., a ribozyme) engineered to cleave a desired endogenous mRNA product. Thus, any gene which produces a protein or mRNA which expresses a phenotype or morphology change of interest is useful for the practice of the present invention. (See also U.S. Pat. No. 7,576,262, “Modified gene-silencing RNA and uses thereof.”)

U.S. Pat. Nos. 7,230,158, 7,122,720, 7,081,363, 6,734,341, 6,503,732, 6,392,121, 6,087,560, 5,981,181, 5,977,060, 5,608,146, 5,516,667, each of which, and all documents cited therein are hereby incorporated herein by reference, consistent with the above INCORPORATION BY REFERENCE section, are additionally cited as examples of U.S. Patents that can concern transformed lettuce and/or methods of transforming lettuce or lettuce plant cells, and techniques from these U.S. Patents, as well as promoters, vectors, etc., can be employed in the practice of this invention to introduce exogenous nucleic acid sequence(s) into a plant of lettuce variety 45-IC7021 RZ, lettuce variety 45-IC7028 RZ, or lettuce variety 45-IC7037 RZ (or cells thereof), and exemplify some exogenous nucleic acid sequence(s) which can be introduced into a plant of lettuce variety 45-IC7021 RZ, lettuce variety 45-IC7028 RZ, or lettuce variety 45-IC7037 RZ (or cells thereof) of the invention, as well as techniques, promoters, vectors etc., to thereby obtain further plants of lettuce variety 45-IC7021 RZ, lettuce variety 45-IC7028 RZ, or lettuce variety 45-IC7037 RZ, plant parts and cells, seeds, other propagation material harvestable parts of these plants, etc. of the invention, e.g. tissue culture, including a cell or protoplast, such as an embryo, meristem, cotyledon, pollen, leaf, anther, root, root tip, pistil, flower, seed or stalk.

The invention further relates to propagation material for producing plants of the invention. Such propagation material can comprise inter alia seeds of the claimed plant and parts of the plant that are involved in sexual reproduction. Such parts are for example selected from the group consisting of seeds, microspores, pollen, ovaries, ovules, embryo sacs and egg cells. In addition, the invention relates to propagation material which can comprise parts of the plant that are suitable for vegetative reproduction, for example cuttings, roots, stems, cells, protoplasts.

According to a further aspect thereof the propagation material of the invention can comprise a tissue culture of the claimed plant. The tissue culture can comprise regenerable cells. Such tissue culture can be derived from leaves, pollen, embryos, cotyledon, hypocotyls, meristematic cells, roots, root tips, anthers, flowers, seeds and stems. (See generally U.S. Pat. No. 7,041,876 on lettuce being recognized as a plant that can be regenerated from cultured cells or tissue).

The invention further relates to a cell of a plant of the invention. Such a cell may either be in isolated form, or a part of the complete plant or parts thereof and still forms a cell of the invention because such a cell comprises the QTLs on chromosome 2, 3 and 7 as defined herein. Each cell of a plant of the invention carries the QTLs on chromosome 2, 3 and 7 as defined herein. A cell of the invention may also be a regenerable cell that can regenerate into a new plant of the invention.

Also, the invention comprehends methods for producing a seed of a “lettuce variety 45-IC7021 RZ-, lettuce variety 45-IC7028 RZ-, or lettuce variety 45-IC7037 RZ”-derived lettuce plant which can comprise (a) crossing a plant of lettuce variety 45-IC7021 RZ, lettuce variety 45-IC7028 RZ, or lettuce variety 45-IC7037 RZ, a sample of seed of which having been deposited under NCIMB Accession No. 44645, 44407, and 44408, respectively, with a second lettuce plant, and (b) whereby seed of a lettuce variety 45-IC7021 RZ-, lettuce variety 45-IC7028 RZ-, or lettuce variety 45-IC7037 RZ-derived lettuce plant form. Such a method can further comprise (c) crossing a plant grown from lettuce variety 45-IC7021 RZ-, lettuce variety 45-IC7028 RZ-, or lettuce variety 45-IC7037 RZ-derived lettuce seed with itself or with a second lettuce plant to yield additional lettuce variety 45-IC7021 RZ-, lettuce variety 45-IC7028 RZ-, or lettuce variety 45-IC7037 RZ-derived lettuce seed, (d) growing the additional lettuce variety 45-IC7021 RZ-, lettuce variety 45-IC7028 RZ-, or lettuce variety 45-IC7037 RZ-derived lettuce seed of step (c) to yield additional lettuce variety 45-IC7021 RZ-, lettuce variety 45-IC7028 RZ-, or lettuce variety 45-IC7037 RZ-derived lettuce plants, and (e) repeating the crossing and growing of steps (c) and (d) for an additional 3-10 generations to further generate lettuce variety 45-IC7021 RZ-, lettuce variety 45-IC7028 RZ-, or lettuce variety 45-IC7037 RZ-derived lettuce plants.

The invention further relates to the above methods that can further comprise selecting at steps b), d), and e), a lettuce variety 45-IC7021 RZ-, lettuce variety 45-IC7028 RZ-, or lettuce variety 45-IC7037 RZ-derived lettuce plant, exhibiting one or more or all of the physiological and morphological characteristics of lettuce variety 45-IC7021 RZ, lettuce variety 45-IC7028 RZ, or lettuce variety 45-IC7037 RZ, a sample of seed of said variety having been deposited under NCIMB Accession No. 44645, 44407, and 44408, respectively, and other selected traits.

In particular, the invention relates to methods for producing a seed of a lettuce variety 45-IC7021 RZ-, lettuce variety 45-IC7028 RZ-, or lettuce variety 45-IC7037 RZ-derived lettuce plant which can comprise (a) crossing a plant of lettuce variety 45-IC7021 RZ, lettuce variety 45-IC7028 RZ, or lettuce variety 45-IC7037 RZ, a sample of seed of which having been deposited under NCIMB Accession No. 44645, 44407, and 44408, with a second lettuce plant and (b) whereby seed of a lettuce variety 45-IC7021 RZ-, lettuce variety 45-IC7028 RZ-, or lettuce variety 45-IC7037 RZ-derived lettuce plant forms, wherein such a method can further comprise (c) crossing a plant grown from lettuce variety 45-IC7021 RZ, lettuce variety 45-IC7028 RZ, or lettuce variety 45-IC7037 RZ-derived lettuce seed with itself or with a second lettuce plant to yield additional lettuce variety 45-IC7021 RZ, lettuce variety 45-IC7028 RZ, or lettuce variety 45-IC7037 RZ-derived lettuce seed, (d) growing the additional lettuce variety 45-IC7021 RZ, lettuce variety 45-IC7028 RZ, or lettuce variety 45-IC7037 RZ-derived lettuce seed of step (c) to yield additional lettuce variety 45-IC7021 RZ, lettuce variety 45-IC7028 RZ, or lettuce variety 45-IC7037 RZ-derived lettuce plants and selecting plants exhibiting one or more or all of the physiological and morphological characteristics of lettuce variety 45-IC7021 RZ, lettuce variety 45-IC7028 RZ, or lettuce variety 45-IC7037 RZ, and (e) repeating the crossing and growing of steps (c) and (d) for an additional 3-10 generations to further generate lettuce variety 45-IC7021 RZ-, lettuce variety 45-IC7028 RZ-, or lettuce variety 45-IC7037 RZ-derived lettuce plants that exhibit one or more or all of the physiological and morphological characteristics of lettuce variety 45-IC7021 RZ, lettuce variety 45-IC7028 RZ, or lettuce variety 45-IC7037 RZ.

The invention additionally provides a method of introducing at least one new trait into a plant of lettuce variety 45-IC7021 RZ, lettuce variety 45-IC7028 RZ, or lettuce variety 45-IC7037 RZ which can comprise: (a) crossing a plant of lettuce variety 45-IC7021 RZ, lettuce variety 45-IC7028 RZ, or lettuce variety 45-IC7037 RZ, a sample of seed of which having been deposited under NCIMB Accession No. 44645, 44407, and 44408, respectively, with a second lettuce plant that can comprise at least one new trait to produce progeny seed; (b) harvesting and planting the progeny seed to produce at least one progeny plant of a subsequent generation, wherein the progeny plant can comprise the at least one new trait; (c) crossing the selected progeny plant with a plant of lettuce variety 45-IC7021 RZ, lettuce variety 45-IC7028 RZ, or lettuce variety 45-IC7037 RZ, to produce backcross progeny seed; (d) harvesting and planting the backcross progeny seed to produce a backcross progeny plant, (e) repeating steps (c) and (d) for at least three additional generations to produce backcross progeny that can comprise the at least one new trait and all of the physiological and morphological characteristics of a plant of lettuce variety 45-IC7021 RZ, lettuce variety 45-IC7028 RZ, or lettuce variety 45-IC7037 RZ, when grown in the same environmental conditions. The invention, of course, includes a lettuce plant produced by this method.

Backcrossing can also be used to improve an inbred plant. Backcrossing transfers a specific desirable trait from one inbred or non-inbred source to an inbred that lacks that trait. This can be accomplished, for example, by first crossing a superior inbred (A) (recurrent parent) to a donor inbred (non-recurrent parent), which carries the appropriate locus or loci for the trait in question. The progeny of this cross are then mated back to the superior recurrent parent (A) followed by selection in the resultant progeny for the desired trait to be transferred from the non-recurrent parent. After five or more backcross generations with selection for the desired trait, the progeny are heterozygous for loci controlling the characteristic being transferred, but are like the superior parent for most or almost all other loci. The last backcross generation would be selfed to give pure breeding progeny for the trait being transferred. When a plant of lettuce variety 45-IC7021 RZ, lettuce variety 45-IC7028 RZ, or lettuce variety 45-IC7037 RZ, a sample of seed of which having been deposited under NCIMB Accession No. 44645, 44407, and 44408, respectively, is used in backcrossing, offspring retaining one or more or all of the physiological and morphological characteristics of lettuce variety 45-IC7021 RZ, lettuce variety 45-IC7028 RZ, or lettuce variety 45-IC7037 RZ are progeny within the ambit of the invention. Backcrossing methods can be used with the present invention to improve or introduce a characteristic into a plant of the invention, being a plant of lettuce variety 45-IC7021 RZ, lettuce variety 45-IC7028 RZ, or lettuce variety 45-IC7037 RZ. See, e.g., U.S. Pat. No. 7,705,206 (incorporated herein by reference consistent with the above INCORPORATION BY REFERENCE section), for a general discussion relating to backcrossing.

The invention further involves a method of determining the genotype of a plant of lettuce variety 45-IC7021 RZ, lettuce variety 45-IC7028 RZ, or lettuce variety 45-IC7037 RZ, a sample of seed of which has been deposited under NCIMB Accession No. 44645, 44407, and 44408, respectively, or a first generation progeny thereof, which can comprise obtaining a sample of nucleic acids from said plant and detecting in said nucleic acids a plurality of polymorphisms. This method can additionally comprise the step of storing the results of detecting the plurality of polymorphisms on a computer readable medium. The plurality of polymorphisms are indicative of and/or give rise to the expression of the physiological and morphological characteristics of lettuce variety 45-IC7021 RZ, lettuce variety 45-IC7028 RZ, or lettuce variety 45-IC7037 RZ.

There are various ways of obtaining genotype data from a nucleic acid sample. Genotype data can be gathered which is specific for certain phenotypic traits (e.g. gene sequences), but also patterns of random genetic variation can be obtained to construct a so-called DNA fingerprint. Depending on the technique used a fingerprint can be obtained that is unique for lettuce variety 45-IC7021 RZ, lettuce variety 45-IC7028 RZ, or lettuce variety 45-IC7037 RZ. Obtaining a unique DNA fingerprint depends on the genetic variation present in a variety and the sensitivity of the fingerprinting technique. A technique known in the art to provide a good fingerprint profile is called AFLP fingerprinting technique (See generally U.S. Pat. No. 5,874,215), but there are many other marker based techniques, such as RFLP (or Restriction fragment length polymorphism), SSLP (or Simple sequence length polymorphism), RAPD (or Random amplification of polymorphic DNA) VNTR (or Variable number tandem repeat), Microsatellite polymorphism, SSR (or Simple sequence repeat), STR (or Short tandem repeat), SFP (or Single feature polymorphism) DArT (or Diversity Arrays Technology), RAD markers (or Restriction site associated DNA markers) (e.g. Baird et al. PloS One Vol. 3 e3376, 2008; Semagn et al. African Journal of Biotechnology Vol. 5 number 25 pp. 2540-2568, 29 December, 2006), cleaved amplified polymorphic sequence (CAPS) markers, and Single Nucleotide Polymorphisms (SNPs). Nowadays, sequence-based methods are utilizing Single Nucleotide Polymorphisms (SNPs) that are randomly distributed across genomes, as a common tool for genotyping (e.g. Elshire et al. PloS One Vol. 6: e19379, 2011; Poland et al. PloS One Vol. 7: e32253; Truong et al. PLoS One Vol. 7 number 5: e37565, 2012). PCR detection of SNPs may for example, be carried out using Kompetitive allele specific PCR (KASP™) technology, which utilizes two labeled fluorogenic oligonucleotide forward primers and an unlabeled common reverse primer (Semagn et al. Mol. Breeding 33:1-14,2013).

With any of the aforementioned genotyping techniques, polymorphisms can be detected when the genotype and/or sequence of the plant of interest is compared to the genotype and/or sequence of one or more reference plants. As used herein, the genotype and/or sequence of a reference plant can be derived from, but is not limited to, any one of the following: parental lines, closely related plant varieties or species, complete genome sequence of a related plant variety or species, or the de novo assembled genome sequence of one or more related plant varieties or species. For example, it is possible to detect polymorphisms for the characteristic of ‘ease of harvest phenotype’ by comparing the genotype and/or the sequence of lettuce variety 45-IC7021 RZ, lettuce variety 45-IC7028 RZ, or lettuce variety 45-IC7037 RZ with the genotype and/or the sequence of one or more reference plants. The reference plant(s) used for comparison in this example can for example be, but is not limited to, the comparison variety Crunchita RZ.

The polymorphism revealed by these techniques can be used to establish links between genotype and phenotype. The polymorphisms can thus be used to predict or identify certain phenotypic characteristics, individuals, or even species. The polymorphisms are generally called markers. It is common practice for the skilled artisan to apply molecular DNA techniques for generating polymorphisms and creating markers.

The polymorphisms of this invention can be provided in a variety of mediums to facilitate use, e.g. a database or computer readable medium, which can also contain descriptive annotations in a form that allows a skilled artisan to examine or query the polymorphisms and obtain useful information.

As used herein “database” refers to any representation of retrievable collected data including computer files such as text files, database files, spreadsheet files and image files, printed tabulations and graphical representations and combinations of digital and image data collections. In a preferred aspect of the invention, “database” refers to a memory system that can store computer searchable information.

As used herein, “computer readable media” refers to any medium that can be read and accessed directly by a computer. Such media include, but are not limited to: magnetic storage media, such as floppy discs, hard disc, storage medium and magnetic tape; optical storage media such as CD-ROM; electrical storage media such as RAM, DRAM, SRAM, SDRAM, ROM; and PROMs (EPROM, EEPROM, Flash EPROM), and hybrids of these categories such as magnetic/optical storage media. A skilled artisan can readily appreciate how any of the presently known computer readable mediums can be used to create a manufacture which can comprise computer readable medium having recorded thereon a polymorphism of the present invention.

As used herein, “recorded” refers to the result of a process for storing information in a retrievable database or computer readable medium. For instance, a skilled artisan can readily adopt any of the presently known methods for recording information on computer readable medium to generate media which can comprise the polymorphisms of the present invention. A variety of data storage structures are available to a skilled artisan for creating a computer readable medium where the choice of the data storage structure will generally be based on the means chosen to access the stored information. In addition, a variety of data processor programs and formats can be used to store the polymorphisms of the present invention on computer readable medium.

The present invention further provides systems, particularly computer-based systems, which contain the polymorphisms described herein. Such systems are designed to identify the polymorphisms of this invention. As used herein, “a computer-based system” refers to the hardware, software and memory used to analyze the polymorphisms. A skilled artisan can readily appreciate that any one of the currently available computer-based system are suitable for use in the present invention.

Lettuce leaves are sold in packaged form, including without limitation as pre-packaged lettuce salad or as lettuce heads. Mention is made of U.S. Pat. No. 5,523,136, incorporated herein by reference consistent with the above INCORPORATION BY REFERENCE section, which provides packaging film, and packages from such packaging film, including such packaging containing leafy produce, and methods for making and using such packaging film and packages, which are suitable for use with the lettuce leaves of the invention. Thus, the invention comprehends the use of and methods for making and using the leaves of the lettuce plant of the invention, as well as leaves of lettuce plants derived from the invention. The invention further relates to a container which can comprise one or more plants of the invention, or one or more lettuce plants derived from a plant of the invention, in a growth substrate for harvest of leaves from the plant in a domestic environment. This way the consumer can pick very fresh leaves for use in salads. More generally, the invention includes one or more plants of the invention or one or more plants derived from lettuce of the invention, wherein the plant is in a ready-to-harvest condition, including with the consumer picking his own, and further including a container which can comprise one or more of these plants.

The invention is illustrated by the following examples:

### EXAMPLES

### Example 1: Measurement of the Head Height Width Ratio

The height (H) and the width (W) of harvested heads of Calesita RZ (crunchy), Crunchita RZ (crunchy), Evionas RZ (iceberg), Scandinas RZ (iceberg), Rictus (cos) and three different iceberg lines with the ease of harvest phenotype (Line 1 (NCIMB 44645), Line 2 (NCIMB 44407) and Line 3 (NCIMB 44408)) grown under the same conditions were measured. The plants were grown outdoor under long-day conditions: sowing 75-85 days before the longest day in a mild mid-latitude climate with a warm summer (Koppen-classification: Csb or Cfb; McKnight & Hess, 2000. Physical Geography: A Landscape Appreciation. Upper Saddle River, NJ: Prentice Hall). The core within such a head should not exceed 8 cm. The measurements were taken when the plants were at mature stage and when the heads had a weight of at least 400 grams. The width corresponds to the largest width of the head and the height corresponds to highest height of the head. Based on the measurements the height/width ratio was measured. Table 8 shows the average of the measurements: iceberg plants have a height/width ratio of 1.0 and 0.9 (average 0.9), ease of harvest iceberg plants have a height/width ratio between 1.2 and 1.4 (average 1.30), crunchy lettuce plants have a height/width ratio between 2.1 and 2.4 (average 2.2) and cos lettuce plants have a height/width ratio of 2.6.

The results show that iceberg lettuce plants and ease of harvest iceberg plants have a height/width ratio between 0.9 and 1.4 which is lower than the ratio of cos lettuce and crunchy lettuce plants when the plants are grown under the same conditions.

TABLE 8
Measurements of the height and width in
centimeters and height/width (H/W) ratio

| | Number | Average | Average | Average |
|---|---|---|---|---|
| | of plants | of W | of H | H/W |
| Crunchy lettuce | 25 | 10.4 | 23.1 | 2.2 |
| Calesita RZ | 15 | 10.1 | 21.5 | 2.1 |
| Crunchita RZ | 10 | 10.7 | 25.5 | 2.4 |
| Iceberg | 25 | 12.6 | 11.8 | 0.9 |
| Evionas RZ | 10 | 12.8 | 12.3 | 1.0 |
| Scandinas RZ | 15 | 12.4 | 11.4 | 0.9 |
| Ease of harvest | 77 | 12.5 | 16.1 | 1.3 |
| iceberg | | | | |
| Line 1 | 27 | 12.9 | 15.5 | 1.2 |
| Line 2 | 25 | 12.1 | 16.9 | 1.4 |
| Line 3 | 25 | 12.4 | 16.1 | 1.3 |
| Cos lettuce | 24 | 9.6 | 24.7 | 2.6 |
| Rictus | 24 | 9.6 | 24.7 | 2.6 |
| Total | 151 | | | |

### Example 2: Detachment of the Outer Leaf

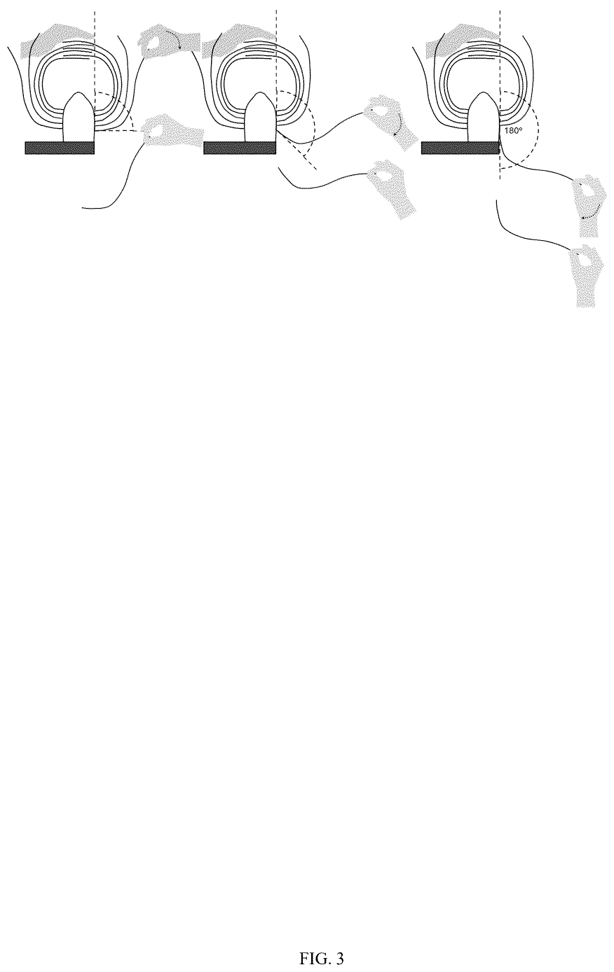

The lettuce head was cut from the stem at mature stage with a head weight of at least 400 grams of plants grown under the same conditions. The plants were grown outdoor under long-day conditions: sowing 75-85 days before the longest day in a mild mid-latitude climate with a warm summer (Koppen-classification: Csb or Cfb; McKnight & Hess, 2000. Physical Geography: A Landscape Appreciation. Upper Saddle River, NJ: Prentice Hall). The core within such a head should not exceed 8 cm. The whole head was held in upright position. The measurement was performed with the 12th oldest leaf which is the 12th leaf counting from the roots to the top of the plant. The 12th leaf was hold by two fingers at 20 cm from the stem and bend until it detached from the stem completely at the point where it is attached to the stem before reaching 180 degrees. The movement to bend the leaf is circular and downward i.e leaves are bent in the direction to the cut stem away from the head of the lettuce plant as represented in FIG. 3.

The 12th outer leaf of the 12 tested iceberg lettuce heads of the variety Scandinas RZ didn't detach i.e. the probability of successful detachment was 0%. For iceberg lettuce plants with the QTLs on chromosome 2, 3 and 7 as defined herein, successful detachment of the 12th leaf was observed. The results show that iceberg plants with the QTLs on chromosome 2, 3 and 7 as defined herein had a 12th that detached as it is required for the ease of harvest phenotype.

### Example 3: QTL Mapping and Marker Development

The first mapping population was derived from a cross between an iceberg lettuce plant and a crunchy lettuce variety. 405 F2 plants were phenotyped for traits that are relevant for the ease of harvest phenotype of an iceberg lettuce plant. DNA was extracted from young leaf tissue from the F2 plants. 78 polymorph KASP markers were run for mapping distributed with an average spacing of 14.8 cM. 12 markers were removed due to segregation distortion. QTL analysis was performed, and mapping of the data resulted in the identification of a QTL on chromosome 2 and a QTL on chromosome 7.

An additional population was developed from a cross between an iceberg lettuce with the ease of harvest phenotype and the iceberg lettuce Kyoto RZ. 250 F2 plants were phenotyped for traits that are relevant for the ease of harvest phenotype of an iceberg lettuce plant. DNA was extracted from young leaf tissue from the F2 plants. 391 polymorph KASP markers were run for mapping distributed with an average spacing of 2.8 cM. The mapping of the data resulted in the identification of a QTL on chromosome 3 and the confirmation of the QTL on chromosome 2.

A third population was developed from a cross between a crunchy lettuce variety and an iceberg lettuce plant. 260 F2 plants were phenotyped for traits that are relevant for the ease of harvest phenotype of an iceberg lettuce plant. DNA was extracted from young leaf tissue from the F2 plants. 312 polymorph KASP markers were run for mapping distributed with an average spacing of 4 cM. The QTLs on chromosome 2, 3 and 7 were confirmed.

The markers developed for the ‘ease of harvest’ phenotype can be found in the sequence tabulation described herein.

### Example 4: Introducing the Ease of Harvest Phenotype in a Lettuce Iceberg Plant

A lettuce iceberg plant comprising the QTLs on chromosome 2, 3 and 7 as defined herein, of which a representative sample of seed was deposited with the NCIMB under accession numbers NCIMB 44645, NCIMB 44407, and NCIMB 44408 was crossed with a plant of the iceberg variety Scandinas RZ to obtain an F1 generation. The resulting F1 from this cross had the same phenotype as Scandinas RZ i.e. the 12^{th} leaf didn't detach from the stem.

From the F1 population a plant was selected which was selfed to obtain a population of F2 plants. The F2 segregates in a manner that corresponds with a recessive inheritance of the trait. Genomic DNA of each plant of the F2 population was isolated and the markers as described in Example 3 were tested.

Plants comprising the QTLs on chromosome 2, 3 and 7 as defined herein homozygously were assayed as described in Example 2 for the presence of the ease of harvest phenotype. A plant having the QTLs on chromosome 2, 3 and 7 as defined herein and the phenotype of the invention was thereby selected.

Plants comprising the QTL on chromosome 3 or on chromosome 2 homozygously were selfed to obtain a F3 population. Genomic DNA of each plant of the F3 population was isolated and the markers as described in Example 3 were tested. Plants comprising the QTLs on chromosome 2, 3 and 7 homozygously were assayed as described in Example 2 for the presence of the ease of harvest phenotype. A plant having the QTLs on chromosome 2, 3 and 7 as defined herein and the phenotype of the invention was thereby selected.

Alternatively, a plant of the F1 population was selected and backcrossed with a plant of the deposits in order to obtain a F2 population. Genomic DNA of each plant of the F2 population was isolated and the markers as described in Example 3 were tested. Plants comprising the QTLs on chromosome 2, 3 and 7 homozygously were assayed as described in Example 2 for the presence of the ease of harvest phenotype. A plant having the QTLs on chromosome 2, 3 and 7 as defined herein and the phenotype of the invention was thereby selected.

The invention is further described by the following numbered paragraphs:
1. A seed of lettuce variety 45-IC7021 RZ, lettuce variety 45-IC7028 RZ, or lettuce variety 45-IC7037 RZ, a representative sample of seed of said varieties having been deposited under NCIMB Accession No. 44645, NCIMB Accession No. 44407, or NCIMB Accession No. 44408, respectively.
2. A lettuce plant grown from the seed of paragraph 1.
3. A lettuce plant grown from seed deposited under NCIMB Accession No. 44645, NCIMB Accession No. 44407, or NCIMB Accession No. 44408, respectively. 4. A lettuce plant, or a part thereof, having all the physiological and morphological characteristics of the lettuce plant of paragraph 2.
5. A part of the lettuce plant of paragraph 2, wherein said part comprises a leaf, embryo, cotyledon, hypocotyl, meristematic cell, root tip, anther, flower, microspore, pollen, ovary, ovule, embryo sac, egg cell, cutting, root, stem, cell or protoplast.
6. A tissue culture of regenerable cells or protoplasts from the lettuce plant of paragraph 2.
7. A method of vegetatively propagating a plant of lettuce variety 45-IC7021 RZ, lettuce variety 45-IC7028 RZ, or lettuce variety 45-IC7037 RZ comprising (a) collecting tissue capable of being propagated from a lettuce plant of paragraph 2, (b) cultivating the tissue to obtain proliferated shoots and rooting the proliferated shoots to obtain rooted plantlets, and (c) optionally growing plants from the rooted plantlets.
8. A method for producing a progeny plant of lettuce variety 45-IC7021 RZ, lettuce variety 45-IC7028 RZ, or lettuce variety 45-IC7037 RZ, comprising crossing a lettuce plant of paragraph 2 with itself or with another lettuce plant, harvesting the resultant seed, and growing said seed.
9. A method for producing a modified lettuce plant comprising mutagenizing the seed of paragraph 1 and growing said seed.
10. A method for producing a modified lettuce plant comprising mutagenizing the plant of paragraph 2.
11. A method for producing a modified lettuce plant comprising mutagenizing the plant part of paragraph 4.
12. A method for producing a modified lettuce plant comprising mutagenizing the tissue culture of paragraph 6.
13 A method of producing a lettuce seed comprising crossing a male parent lettuce plant with a female parent lettuce plant and harvesting the resultant lettuce seed, wherein said male parent lettuce plant or said female parent lettuce plant is the lettuce plant of paragraph 2.
14. A F1 lettuce seed produced by the method of paragraph 13.
15. A method for producing a seed of a lettuce variety 45-IC7021 RZ-derived, lettuce variety 45-IC7028 RZ-derived, or lettuce variety 45-IC7037 RZ-derived lettuce plant comprising (a) crossing a plant of lettuce variety 45-IC7021 RZ, lettuce variety 45-IC7028 RZ, or lettuce variety 45-IC7037 RZ, a representative sample of seed of which having been deposited under NCIMB Accession No. 44645, NCIMB Accession No. 44407, and NCIMB Accession No. 44408, respectively, with a second lettuce plant, and (b) whereby seed of a lettuce variety 45-IC7021 RZ-derived, lettuce variety 45-IC7028 RZ-derived, or lettuce variety 45-IC7037 RZ-derived lettuce plant forms.
16. The method of paragraph 15 further comprising (c) crossing a plant grown from 45-IC7021 RZ-derived, lettuce variety 45-IC7028 RZ-derived, or lettuce variety 45-IC7037 RZ-derived lettuce seed with itself or with a second lettuce plant to yield additional 45-IC7021 RZ-derived, lettuce variety 45-IC7028 RZ-derived, or lettuce variety 45-IC7037 RZ-derived lettuce seed, (d) growing the additional 45-IC7021 RZ-derived, lettuce variety 45-IC7028 RZ-derived, or lettuce variety 45-IC7037 RZ-derived lettuce seed of step (c) to yield additional 45-IC7021 RZ-derived, lettuce variety 45-IC7028 RZ-derived, or lettuce variety 45-IC7037 RZ-derived lettuce plants, and (e) repeating the crossing and growing of steps (c) and (d) for an additional 3-10 generations to generate further 45-IC7021 RZ-derived, lettuce variety 45-IC7028 RZ-derived, or lettuce variety 45-IC7037 RZ-derived lettuce plants, and (f) whereby seed of a 45-IC7021 RZ-derived, lettuce variety 45-IC7028 RZ-derived, or lettuce variety 45-IC7037 RZ-derived lettuce plant forms.
17. A method of introducing at least one new trait into a plant of 45-IC7021 RZ, lettuce variety 45-IC7028 RZ, or lettuce variety 45-IC7037 RZ comprising: (a) crossing a plant of lettuce variety 45-IC7021 RZ, lettuce variety 45-IC7028 RZ, or lettuce variety 45-IC7037 RZ, a representative sample of seed of which having been deposited under NCIMB Accession No. 44645, NCIMB Accession No. 44407, and NCIMB Accession No. 44408, respectively, with a second lettuce plant that comprises at least one new trait to produce progeny seed, (b) harvesting and planting the progeny seed to produce at least one progeny plant of a subsequent generation, wherein the progeny plant comprises the at least one new trait, (c) crossing the progeny plant with a plant of lettuce variety 45-IC7021 RZ, lettuce variety 45-IC7028 RZ, or lettuce variety 45-IC7037 RZ to produce backcross progeny seed, (d) harvesting and planting the backcross progeny seed to produce a backcross progeny plant, and (e) repeating steps (c) and (d) for at least three additional generations to produce a lettuce plant of variety 45-IC7021 RZ, variety 45-IC7028 RZ, or 45-IC7037 RZ comprising at least one new trait and all of the physiological and morphological characteristics of a plant of lettuce variety 45-IC7021 RZ, variety 45-IC7028 RZ, or variety 45-IC7037 RZ, when grown in the same environmental conditions.
18. The lettuce plant produced by the method of paragraph 17, wherein the plant comprises the at least one new trait and otherwise all of the physiological and morphological characteristics of a lettuce plant of variety 45-IC7021 RZ, variety 45-IC7028 RZ, or variety 45-IC7037 RZ.
19. A method for producing lettuce leaves as a food product comprising sowing the seed of paragraph 1 and growing the seed into a harvestable lettuce plant and harvesting the head or leaves of said plant, processing and/or packaging the head or the leaves.
20. A container comprising one or more lettuce plants of paragraph 2 or heads thereof for harvest of leaves.

Having thus described in detail preferred embodiments of the present invention, it is to be understood that the invention is not to be limited to particular details set forth in the above description as many apparent variations thereof are possible without departing from the spirit or scope of the present invention.

What is claimed is:

1. A seed of lettuce variety 45-IC7021 RZ, lettuce variety 45-IC7028 RZ, or lettuce variety 45-IC7037 RZ, a representative sample of seed of said varieties having been deposited under NCIMB Accession No. 44645, NCIMB Accession No. 44407, or NCIMB Accession No. 44408, respectively.

2. A lettuce plant grown from the seed of claim 1.

3. A lettuce plant grown from seed deposited under NCIMB Accession No. 44645, NCIMB Accession No. 44407, or NCIMB Accession No. 44408, respectively.

4. A lettuce plant, or a part thereof, having all the physiological and morphological characteristics of the lettuce plant of claim 2.

5. A part of the lettuce plant of claim 2, wherein said part comprises a leaf, embryo, cotyledon, hypocotyl, meristematic cell, root tip, anther, flower, microspore, pollen, ovary, ovule, embryo sac, egg cell, cutting, root, stem, cell or protoplast.

6. A tissue culture of regenerable cells or protoplasts from the lettuce plant of claim 2.

7. A method of vegetatively propagating a plant of lettuce variety 45-IC7021 RZ, lettuce variety 45-IC7028 RZ, or lettuce variety 45-IC7037 RZ comprising (a) collecting tissue capable of being propagated from a lettuce plant of claim 2, (b) cultivating the tissue to obtain proliferated shoots and rooting the proliferated shoots to obtain rooted plantlets, and (c) optionally growing plants from the rooted plantlets.

8. A method for producing a progeny plant of lettuce variety 45-IC7021 RZ, lettuce variety 45-IC7028 RZ, or lettuce variety 45-IC7037 RZ, comprising crossing a lettuce plant of claim 2 with itself or with another lettuce plant, harvesting the resultant seed, and growing said seed.

9. A method for producing a modified lettuce plant comprising mutagenizing the seed of claim 1 and growing said seed.

10. A method for producing a modified lettuce plant comprising mutagenizing the plant of claim 2.

11. A method for producing a modified lettuce plant comprising mutagenizing the plant part of claim 4.

12. A method for producing a modified lettuce plant comprising mutagenizing the tissue culture of claim 6.

13. A method of producing a lettuce seed comprising crossing a male parent lettuce plant with a female parent lettuce plant and harvesting the resultant lettuce seed, wherein said male parent lettuce plant or said female parent lettuce plant is the lettuce plant of claim 2.

14. An F1 lettuce seed produced by the method of claim 13.

15. A method for producing a seed of a lettuce variety 45-IC7021 RZ-derived, lettuce variety 45-IC7028 RZ-derived, or lettuce variety 45-IC7037 RZ-derived lettuce plant comprising (a) crossing a plant of lettuce variety 45-IC7021 RZ, lettuce variety 45-IC7028 RZ, or lettuce variety 45-IC7037 RZ, a representative sample of seed of which having been deposited under NCIMB Accession No. 44645, NCIMB Accession No. 44407, and NCIMB Accession No. 44408, respectively, with a second lettuce plant, and (b) whereby seed of a lettuce variety 45-IC7021 RZ-derived, lettuce variety 45-IC7028 RZ-derived, or lettuce variety 45-IC7037 RZ-derived lettuce plant forms.

16. The method of claim 15 further comprising (c) crossing a plant grown from 45-IC7021 RZ-derived, lettuce variety 45-IC7028 RZ-derived, or lettuce variety 45-IC7037 RZ-derived lettuce seed with itself or with a second lettuce plant to yield additional 45-IC7021 RZ-derived, lettuce variety 45-IC7028 RZ-derived, or lettuce variety 45-IC7037 RZ-derived lettuce seed, (d) growing the additional 45-IC7021 RZ-derived, lettuce variety 45-IC7028 RZ-derived, or lettuce variety 45-IC7037 RZ-derived lettuce seed of step (c) to yield additional 45-IC7021 RZ-derived, lettuce variety 45-IC7028 RZ-derived, or lettuce variety 45-IC7037 RZ-derived lettuce plants, and (e) repeating the crossing and growing of steps (c) and (d) for an additional 3-10 generations to generate further 45-IC7021 RZ-derived, lettuce variety 45-IC7028 RZ-derived, or lettuce variety 45-IC7037 RZ-derived lettuce plants, and (f) whereby seed of a 45-IC7021 RZ-derived, lettuce variety 45-IC7028 RZ-derived, or lettuce variety 45-IC7037 RZ-derived lettuce plant forms.

17. A method of introducing at least one new trait into a plant of 45-IC7021 RZ, lettuce variety 45-IC7028 RZ, or lettuce variety 45-IC7037 RZ comprising: (a) crossing a plant of lettuce variety 45-IC7021 RZ, lettuce variety 45-IC7028 RZ, or lettuce variety 45-IC7037 RZ, a representative sample of seed of which having been deposited under NCIMB Accession No. 44645, NCIMB Accession No. 44407, and NCIMB Accession No. 44408, respectively, with a second lettuce plant that comprises at least one new trait to produce progeny seed, (b) harvesting and planting the progeny seed to produce at least one progeny plant of a subsequent generation, wherein the progeny plant comprises the at least one new trait, (c) crossing the progeny plant with a plant of lettuce variety 45-IC7021 RZ, lettuce variety 45-IC7028 RZ, or lettuce variety 45-IC7037 RZ to produce backcross progeny seed, (d) harvesting and planting the backcross progeny seed to produce a backcross progeny plant, and (e) repeating steps (c) and (d) for at least three additional generations to produce a lettuce plant of variety 45-IC7021 RZ, variety 45-IC7028 RZ, or 45-IC7037 RZ comprising at least one new trait and all of the physiological and morphological characteristics of a plant of lettuce variety 45-IC7021 RZ, variety 45-IC7028 RZ, or variety 45-IC7037 RZ, when grown in the same environmental conditions.

18. The lettuce plant produced by the method of claim 17, wherein the plant comprises the at least one new trait and otherwise all of the physiological and morphological characteristics of a lettuce plant of variety 45-IC7021 RZ, variety 45-IC7028 RZ, or variety 45-IC7037 RZ.

19. A method for producing lettuce leaves as a food product comprising sowing the seed of claim 1 and growing the seed into a harvestable lettuce plant and harvesting the head or leaves of said plant, processing and/or packaging the head or the leaves.

20. A container comprising one or more lettuce plants of claim 2 or heads thereof for harvest of leaves.
